(19) United States
(12) Patent Application Publication
(10) Pub. No.: US 2025/0226095 A1
(43) Pub. Date: Jul. 10, 2025

(54) LABEL GENERATION METHOD, LABEL GENERATION DEVICE, TRAINED MODEL GENERATION METHOD, MACHINE LEARNING DEVICE, IMAGE PROCESSING METHOD, IMAGE PROCESSING DEVICE, AND PROGRAM

(71) Applicant: FUJIFILM Corporation, Tokyo (JP)

(72) Inventor: Yuta HIASA, Tokyo (JP)

(73) Assignee: FUJIFILM Corporation, Tokyo (JP)

(21) Appl. No.: 18/986,659

(22) Filed: Dec. 18, 2024

(30) Foreign Application Priority Data
Jan. 4, 2024 (JP) 2024-000354

(51) Int. Cl. G16H 50/20; A61B 5/08; G06T 15/00; G06V 10/46; G06V 10/764; G16H 30/40

(52) U.S. Cl. CPC G16H 50/20; A61B 5/08; G06T 15/00; G06V 10/462; G06V 10/764; G16H 30/40

(57) ABSTRACT

A label generation method enables to provide information on a position of a disease in a medical image and a certainty level thereof corresponding to a severity level, the method comprising causing one or more first processors to: acquire one or more candidate positions of a disease in a first division unit from a first medical image; acquire diagnostic information in which a position of the disease is indefinite or the position of the disease is specified in a second division unit; convert the diagnostic information into a certainty level label corresponding to a severity level of the disease; associate a certainty level of the disease corresponding to the certainty level label with the candidate positions of the disease acquired from the first medical image; and acquire a ground truth label, which is generated by the association, of the position and the certainty level of the disease.

### CROSS-REFERENCE TO RELATED APPLICATION

The present application claims priority under 35 U.S.C § 119(a) to Japanese Patent Application No. 2024-000354 filed on Jan. 4, 2024, which is hereby expressly incorporated by reference, in its entirety, into the present application.

### BACKGROUND OF THE INVENTION

### 1. Field of the Invention

The present disclosure relates to a label generation method, a label generation device, a trained model generation method, a machine learning device, an image processing method, an image processing device, and a program, and particularly relates to an information processing technology contributing to medical image diagnosis support.

### 2. Description of the Related Art

WO2018/225448A describes a method of supporting diagnosis of a disease using an endoscopic image of a digestive organ by using a neural network. The method described in WO2018/225448A is characterized in that the neural network is trained using a first endoscopic image of the digestive organ and a definitive diagnosis result of at least one of a positive or negative diagnosis of a disease of the digestive organ, a past disease, a severity level, or information corresponding to an imaged part, which corresponds to the first endoscopic image, and the trained neural network outputs, based on a second endoscopic image of the digestive organ, at least one of a probability of the positive and/or negative diagnosis of the disease of the digestive organ, a probability of the past disease, the severity level of the disease, or the information corresponding to the imaged part.

### SUMMARY OF THE INVENTION

### Object 1: Importance of Estimation of Severity Level of Disease

In a case in which a position of the disease is estimated from a medical image using machine learning, training data (learning data) in which a specialist has annotated the presence or absence of the disease or the position of the disease on the medical image is generally used. A machine learning model trained using this training data estimates the position and a certainty level of the disease thereof from an input image, but the certainty level in this case is not in accordance with the severity level of the disease and often depends on an appearance frequency of each disease pattern for each disease type. For example, in a case of a model that detects pleural effusion from a chest X-ray image, even in a case of a small amount of pleural effusion, a score of the certainty level is high in a typical example.

Therefore, in an interpretation support system that provides a doctor with the position and the certainty level of the disease of the disease by using such a model, the position of the disease with a low severity level (mild disease) is provided as information with a high certainty level, and information that does not match to the intuition of the doctor who places importance on the severity level (grading) of the disease is provided. In order to achieve such an object, a system that provides diagnosis support information that matches the intuition of the doctor as much as possible is desired.

### Object 2: Importance of Combination of Information Indicating Severity Level of Disease and Information on Disease Position

In order to achieve the image interpretation support system that can provide information on the certainty level in accordance with the severity level of the disease so as to match intuitive understanding of the doctor who places importance on the severity level of the disease, it is conceivable to generate a machine learning model that estimates the position of the disease and the certainty level thereof corresponding to the severity level from the medical image by using the machine learning. In order to generate such a machine learning model, it is necessary to prepare a large number of paired data including a medical image for training and data of a label indicating ground truth of the position of the disease in the medical image and the certainty level of the disease, that is, ground truth data.

In a case of generating the ground truth label of the certainty level corresponding to the severity level of the disease, it is conceivable to use diagnostic information as information indicating the severity level of the disease. The diagnostic information includes information obtained by a definitive diagnosis examination (hereinafter, referred to as definitive diagnosis examination information). WO2018/225448A describes training the neural network using the severity level which is the definitive diagnosis result for the first endoscopic image, but the definitive diagnosis result in WO2018/225448A is based on the premise that the data includes information on an anatomical imaging part such as a “pharynx” or an “esophagus”.

However, there is also data in which the disease position is not clearly indicated in the diagnostic information. For example, since sputum examination information is a measurement value obtained by measuring a total amount of bacteria discharged from a lung, it is not possible to specify where (at which position) the disease is present in the lung. In a case in which the machine learning model is trained using data in which the position of the disease is indefinite, it is possible to estimate the severity level of the disease, but it is not easy to identify the position of the disease. The technology of WO2018/225448A cannot be applied to data in which the disease position is indefinite in the diagnostic information.

Alternatively, even in a case of the diagnostic information in which the disease position is recorded, there may be data in which region division granularity of the position information is not desired granularity. For example, while there is data in which the position of the disease is recorded in units of region division based on an anatomical structure, such as a name of a part of an organ, as the diagnostic information, a task to be achieved by the machine learning model is processing of estimating the position and the certainty level of the disease in units of pixels from the input medical image, and the region division granularity of the position may be different. Even in such a case, the technology of WO2018/225448A cannot be applied, and it is difficult to generate the machine learning model that achieves a target task.

The present disclosure has been made in view of such circumstances, and an object of the present disclosure is to provide information that matches the intuition of a doctor as much as possible by performing machine learning using a position of a disease and a certainty level thereof corresponding to a severity level in a medical image. In relation to this object, an object of the present disclosure is to provide a label generation method, a label generation device, and a program capable of efficiently generating a ground truth label that can contribute to the generation of a machine learning model that estimates the position of the disease and the certainty level thereof corresponding to the severity level from the medical image.

Another object of the present disclosure is to provide a trained model generation method, a machine learning device, and a program for performing machine learning using the ground truth label generated by the label generation method according to the aspect of the present disclosure. Still another object of the present disclosure is to provide an image processing device and a program capable of generating information indicating the position and the certainty level of the disease in the medical image by using a trained machine learning model and provide the information in a form that is easy for the doctor to intuitively understand.

A first aspect of the present disclosure relates to a label generation method comprising: causing one or more first processors to execute: a step of acquiring one or more candidate positions of a disease in a first division unit from a first medical image; a step of acquiring diagnostic information, for the first medical image, in which a position of the disease is indefinite or the position of the disease is specified in a second division unit; a step of converting the diagnostic information into a certainty level label corresponding to a severity level of the disease; a step of associating a certainty level of the disease corresponding to the certainty level label with the candidate positions of the disease acquired from the first medical image; and a step of acquiring a ground truth label, which is generated by the association, of the position and the certainty level of the disease with respect to the first medical image.

According to the first aspect, by combining the candidate positions of the disease obtained from the first medical image and the severity level of the disease understood from the diagnostic information to convert the severity level into the certainty level label and associating the certainty level corresponding to the severity level of the disease with the candidate positions of the disease, the ground truth label of the position and the certainty level of the disease with respect to the first medical image can be generated.

The first division unit and the second division unit for defining the fineness (granularity) of the information indicating the position may be different division units. The term “division unit” means a unit for dividing a region in order to distinguish the positions. According to the first aspect, it is possible to efficiently generate the ground truth label by using the diagnostic information in which the position of the disease is indefinite or the diagnostic information in which the division unit of the position of the disease is different from a desired division unit.

A second aspect relates to the label generation method according to the first aspect, in which in the step of acquiring the ground truth label, the one or more first processors may acquire the ground truth label of the position and the certainty level of the disease in the first division unit or the second division unit.

A third aspect relates to the label generation method according to the first or second aspect, which may further comprise: causing the one or more first processors to execute: a step of acquiring anatomical structure information from the first medical image, in which in the step of associating the certainty level of the disease with the candidate positions of the disease, the position of the disease may be constrained to be located within a desired anatomical structure specified from the anatomical structure information.

A fourth aspect relates to the label generation method according to any one of the first to third aspects, in which the diagnostic information may be a three-dimensional examination image, and the step of converting the diagnostic information into the certainty level label may include a step of recognizing an anatomical structure from the three-dimensional examination image, a step of recognizing the position of the disease from the three-dimensional examination image, and a step of calculating the certainty level label of the disease for each anatomical structure from the recognized anatomical structure and the recognized position of the disease.

A fifth aspect relates to the label generation method according to any one of the first to third aspects, in which the diagnostic information may be sputum examination information including an examination result of a sputum examination, and the step of converting the diagnostic information into the certainty level label may include a step of calculating the certainty level label of the disease based on an amount of bacteria collected in the sputum examination.

A sixth aspect relates to the label generation method according to any one of the first to fifth aspects, in which in the step of acquiring the one or more candidate positions of the disease, a saliency map of the disease may be calculated by using a first machine learning model that has been trained in advance.

A seventh aspect relates to the label generation method according to the sixth aspect, in which in the step of associating the certainty level of the disease with the candidate positions of the disease, the certainty level label may be weighted by a value of the saliency map.

An eighth aspect relates to the label generation method according to any one of the first to seventh aspects, in which the diagnostic information may be information in which the position of the disease is specified in the second division unit, the label generation method may further comprise: causing the one or more first processors to execute: a step of acquiring anatomical structure information in a third division unit from the first medical image; a step of converting the candidate positions of the disease in the first division unit into candidate positions of the disease in the third division unit; and a step of converting a certainty level label in the second division unit converted from the diagnostic information into a certainty level label in the third division unit, in the step of associating the certainty level of the disease with the candidate positions of the disease, a certainty level of the disease corresponding to the certainty level label in the third division unit may be associated with the candidate positions of the disease in the third division unit, and in the step of acquiring the ground truth label, the ground truth label of the position and the certainty level of the disease may be acquired in the third division unit.

A ninth aspect relates to the label generation method according to any one of the first to eighth aspects, in which the first medical image may be a chest X-ray image, a computed tomography image, or a magnetic resonance image.

A tenth aspect relates to the label generation method according to any one of the first to ninth aspects, in which at least one of pleural effusion, pneumothorax, or pulmonary tuberculosis may be targeted as the disease.

An eleventh aspect of the present disclosure relates to a trained model generation method comprising: causing one or more second processors to execute: a step of training a second machine learning model through machine learning using training data including the ground truth label generated by the label generation method according to any one of the first to tenth aspects, in which the trained second machine learning model is generated, which has been trained to receive an input of a second medical image and output the position and the certainty level of the disease with respect to the second medical image.

A twelfth aspect relates to the trained model generation method according to the eleventh aspect, in which the certainty level label of the disease is represented by a continuous value, and in the step of training the second machine learning model, the certainty level of the disease may be regression-predicted from the first medical image by the second machine learning model.

A thirteenth aspect relates to the trained model generation method according to the eleventh aspect, in which the certainty level label of the disease may be represented by a discrete value, and in the step of training the second machine learning model, the certainty level of the disease may be classification-predicted from the first medical image by the second machine learning model.

A fourteenth aspect of the present disclosure relates to an image processing method comprising: causing one or more third processors to execute: a step of calculating, by using the trained second machine learning model generated by the trained model generation method according to any one of the eleventh to thirteenth aspects, the position and the certainty level of the disease with respect to the second medical image.

A fifteenth aspect relates to the image processing method according to the fourteenth aspect, which may further comprise: causing the one or more third processors to execute: a step of changing a display form of the disease in accordance with a value of the certainty level of the disease with respect to the second medical image.

A sixteenth aspect of the present disclosure relates to a label generation device comprising: one or more first processors, in which the one or more first processors execute: processing of acquiring one or more candidate positions of a disease in a first division unit from a first medical image; processing of acquiring diagnostic information, for the first medical image, in which a position of the disease is indefinite or the position of the disease is specified in a second division unit; processing of converting the diagnostic information into a certainty level label corresponding to a severity level of the disease; processing of associating a certainty level of the disease corresponding to the certainty level label with the candidate positions of the disease acquired from the first medical image; and processing of acquiring a ground truth label, which is generated by the processing of associating, of the position and the certainty level of the disease with respect to the first medical image.

A seventeenth aspect of the present disclosure relates to a machine learning device comprising: one or more second processors, in which the one or more second processors execute processing of training a second machine learning model through machine learning using training data including the ground truth label generated by the label generation method according to any one of the first to tenth aspects, and the second machine learning model is trained such that the second machine learning model receives an input of a second medical image and outputs the position and the certainty level of the disease in the second medical image.

An eighteenth aspect of the present disclosure relates to an image processing device comprising: one or more third processors, in which the one or more third processors execute processing of calculating, by using the trained second machine learning model generated by the trained model generation method according to any one of the eleventh to thirteenth aspects, the position and the certainty level of the disease with respect to the second medical image.

A nineteenth aspect of the present disclosure relates to a program for causing a computer to execute the label generation method according to any one of the first to tenth aspects.

A twentieth aspect of the present disclosure relates to a program for causing a computer to execute the trained model generation method according to the first to thirteenth aspects.

A twenty-first aspect of the present disclosure relates to a program for causing a computer to execute the image processing method according to the fourteenth or fifteenth aspect.

With the label generation method, the label generation device, and the program according to the present disclosure, it is possible to efficiently generate the ground truth label that can contribute to the generation of the machine learning model that estimates the position of the disease and the certainty level thereof corresponding to the severity level from the medical image. In addition, with the trained model generation method, the machine learning device, and the program according to the present disclosure, it is possible to generate the trained machine learning model that estimates the position of the disease and the certainty level corresponding to the severity level of the disease from the medical image through the machine learning using the generated ground truth label. Further, with the image processing method, the image processing device, and the program according to the present disclosure, it is possible to provide the information indicating the position and the certainty level of the disease in the medical image in a form of information that is easy for the doctor to intuitively understand, by using the generated trained machine learning model.

### BRIEF DESCRIPTION OF THE DRAWINGS

FIG. 1 is a schematic block diagram schematically showing an overall configuration example of a system including a label generation device, a machine learning device, and an image processing device according to an embodiment of the present disclosure.

FIG. 2 is a block diagram showing an example of a hardware configuration of the label generation device according to the embodiment.

FIG. 3 is an explanatory diagram showing Example 1 of a label generation method executed by the label generation device.

FIG. 4 is a block diagram schematically showing a functional configuration of the label generation device that executes the label generation method shown in FIG. 3.

FIG. 5 is a flowchart showing Example 1 of the label generation method.

FIG. 6 is an explanatory diagram showing Example 2 of the label generation method executed by the label generation device.

FIG. 7 is an explanatory diagram showing Example 3 of the label generation method executed by the label generation device.

FIG. 8 is a block diagram schematically showing a functional configuration of the label generation device that executes the label generation method shown in FIG. 7.

FIG. 9 is a flowchart showing Example 3 of the label generation method.

FIG. 10 is a block diagram showing an example of a program and data stored in a memory of the label generation device that executes the label generation method shown in FIG. 9.

FIG. 11 is a block diagram showing an example of a hardware configuration of the machine learning device according to the embodiment.

FIG. 12 is a block diagram schematically showing a functional configuration of the machine learning device.

FIG. 13 is a flowchart showing an example of a machine learning method executed by the machine learning device.

FIG. 14 is a block diagram showing an example of a hardware configuration of the image processing device according to the embodiment.

FIG. 15 is a block diagram schematically showing a functional configuration of the image processing device.

FIG. 16 is an explanatory diagram showing an example of an image processing method executed by using a third machine learning model implemented in the image processing device.

FIG. 17 shows an example of a composite image displayed on a display device as a processing result of the third machine learning model.

FIG. 18 shows an example of the composite image displayed on the display device as the processing result of the third machine learning model.

FIG. 19 shows a display example of an examination list that provides information on a severity level of a disease indicated by a certainty level calculated by the third machine learning model.

### DESCRIPTION OF THE PREFERRED EMBODIMENTS

Hereinafter, detailed description of a preferred embodiment of the present invention will be made with reference to the accompanying drawings.

### Overall Configuration Example of System According to Embodiment

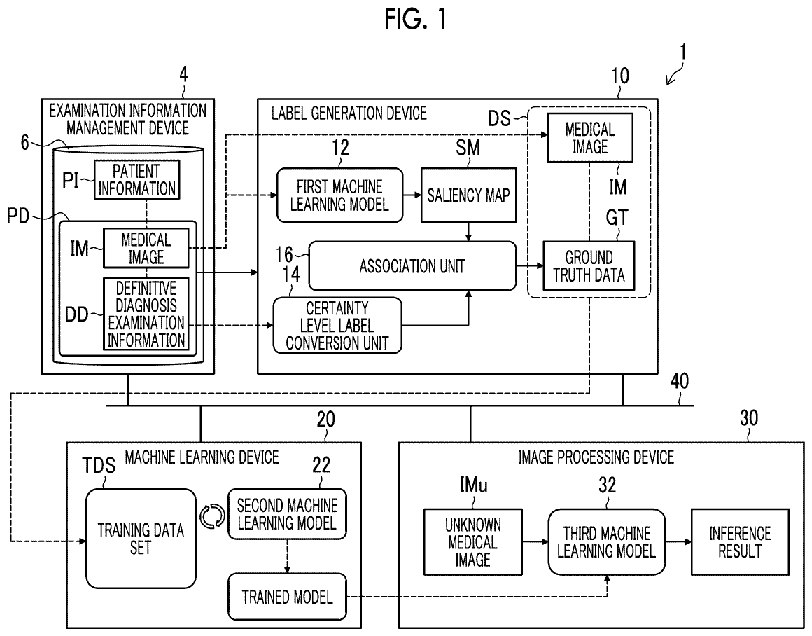

FIG. 1 is a block diagram schematically showing an overall configuration example of a system 1 according to the embodiment of the present disclosure. The system 1 includes an examination information management device 4, a label generation device 10, a machine learning device 20, and an image processing device 30. Processing functions of these devices can be achieved by a combination of hardware and software of a computer.

The examination information management device 4 is an information processing device which stores and manages information including examination results of various examinations performed in a medical facility. The examination information management device 4 comprises a large-capacity storage device 6 and a database management program. The storage device 6 stores various types of data including a medical image IM captured using a modality apparatus. The modality apparatus may be, for example, various examination apparatuses such as an X-ray imaging apparatus, a computed tomography (CT) apparatus, a magnetic resonance imaging (MRI) apparatus, an ultrasound diagnostic apparatus, a positron emission tomography (PET) apparatus, a mammography apparatus, an X-ray fluoroscopy diagnostic apparatus, and an endoscope apparatus.

The examination information management device 4 may function as a medical image management system (picture archiving and communication system: PACS). The examination information management device 4 may include, for example, a digital imaging and communications in medicine (DICOM) server that operates in accordance with specifications of DICOM.

In the storage device 6, the medical images IM for a plurality of patients and definitive diagnosis examination information DD are stored in a state of being linked with patient information PI. The “link” is synonymous with “associate”. The medical image IM may be, as an example, a plain chest X-ray image. The definitive diagnosis examination may be, as an example, a CT examination. In a case in which a target disease is pleural effusion, the definitive diagnosis examination information DD may be, for example, a pleural effusion amount for each of left and right lung fields measured from the CT image, may be a CT image which is an examination image, or may be an MRI image. Alternatively, the definitive diagnosis examination may be a sputum examination, and the definitive diagnosis examination information DD in this case may be an amount of bacteria indicating the examination result of the sputum examination. The sputum examination information is an example of data in which the disease position is indefinite.

The label generation device 10 is an information processing device that acquires the medical image IM stored in the storage device 6 and the definitive diagnosis examination information DD corresponding to the medical image IM, and generates a ground truth label of the position of the disease and a certainty level corresponding to a severity level with respect to the medical image IM, based on paired data PD.

The label generation device 10 includes a first machine learning model 12 that has been trained in advance, a certainty level label conversion unit 14, and an association unit 16. The first machine learning model 12 is a disease detection model that has been trained (subjected to learning) through machine learning so as to estimate candidate positions of the disease from the input medical image IM.

The first machine learning model 12 may be, for example, a model that executes a segmentation task of recognizing the disease from the medical image IM and performing labeling in units of pixels. The first machine learning model 12 is configured by, for example, a neural network. The first machine learning model 12 may be configured by a convolutional neural network. It should be noted that the first machine learning model 12 is a program in substance.

The label generation device 10 can acquire a saliency map SM indicating the candidate positions of the disease in the medical image IM from the output of the first machine learning model 12 with respect to the input of the medical image IM. The saliency map SM may be a binary image or a heat map image in which the candidate positions of the disease are visualized. The granularity of the candidate positions of the disease shown in the saliency map SM may be, for example, a unit of pixels of the medical image IM.

The “granularity” for the information indicating the position is the fineness of a unit for dividing a target region in order to specify the position, and means region division granularity. Small (fine) granularity means that a region as one unit of the region division is small. In the present specification, a region as the unit for region division is referred to as a “division unit”. The term “region division granularity” can be understood by being replaced with the term “division unit”.

The definitive diagnosis examination information DD may be data in which the disease position is indefinite, or data in which the disease position is recorded. In a case in which the information on the disease position is included in the definitive diagnosis examination information DD, the granularity of the information indicating the disease position, that is, the region division granularity may be granularity coarser than the region division granularity of the saliency map SM. For example, the region division granularity of the disease position recorded in the definitive diagnosis examination information DD may be a division unit of an anatomical structure, such as the right lung field or the left lung field. The term “anatomical structure” means an anatomy structure.

The certainty level label conversion unit 14 performs processing of converting the severity level of the disease understood from the definitive diagnosis examination information DD into a certainty level label. The “severity level” may be rephrased as grading of the disease. The certainty level label may be defined by a discrete value or a continuous value.

The association unit 16 is a processing unit that associates the position of the disease with the certainty level. The association unit 16 associates the certainty level indicating the severity level for each candidate position of the disease based on the candidate positions of the disease specified by the saliency map SM and the certainty level label calculated by the certainty level label conversion unit 14. In the processing of the association unit 16, a ground truth label of the position and the certainty level of the disease with respect to the medical image IM is generated.

The label generation device 10 generates ground truth data GT that is label data to which the ground truth label is assigned (associated) for each position specified at a predetermined region division granularity in the medical image IM.

The label generation device 10 generates corresponding ground truth data GT for each of a plurality of medical images IM, and generates a data set DS including a plurality of sets of paired data of the medical image IM and the ground truth data GT. A part or all of the data sets DS generated in this way are used as a training data set TDS for machine learning.

The machine learning device 20 is a computer system that performs machine learning using the training data set TDS to train a second machine learning model 22. The second machine learning model 22 is trained to receive the input of the medical image IM included in the training data set TDS and output the position and the certainty level of the disease in the medical image IM.

The machine learning device 20 updates parameters of the second machine learning model 22 such that the output from the second machine learning model 22 with respect to the input of the medical image IM is close to the ground truth data GT. The second machine learning model 22 is configured by, for example, a neural network. The second machine learning model 22 may be configured by a convolutional neural network. The machine learning device 20 may have a configuration in which the parameters of the second machine learning model 22 are optimized by, for example, a deep learning algorithm.

The machine learning device 20 executes the machine learning using the training data set TDS, and thus the second machine learning model 22 is generated, which has trained (subjected to learning) and has a desired inference performance. A third machine learning model 32, which is the trained model generated in this way, is implemented in the image processing device 30. It should be noted that the second machine learning model 22 and the third machine learning model 32 are programs in substance.

The image processing device 30 is an information processing device (computer system) that comprises the third machine learning model 32, that receives the input of an unknown medical image IMu, that infers the position and the certainty level of the disease with respect to the unknown medical image IMu by using the third machine learning model 32, and that outputs an inference result. The image processing device 30 may be incorporated as, for example, a part of an image interpretation support system.

The examination information management device 4, the label generation device 10, the machine learning device 20, and the image processing device 30 may be connected to each other via an electric communication line 40 so as to be communicable to each other, or a part or all of these devices may be configured as stand-alone devices. The data transfer between the devices is not limited to being performed via the network, and for example, a portable information recording medium may be used. The electric communication line 40 may be a wide area communication line, a premises communication line, or a combination thereof.

In FIG. 1, the examination information management device 4, the label generation device 10, the machine learning device 20, and the image processing device 30 are shown as separate devices, but the processing functions of a plurality of devices among these devices can also be integrated into one device.

For example, the storage device 6 of the examination information management device 4 may be included in the label generation device 10. Further, for example, the label generation device 10 and the machine learning device 20 may be integrated to be configured as one device. The processing functions of the examination information management device 4, the label generation device 10, the machine learning device 20, and the image processing device 30 can be achieved by a computer system including one or a plurality of computers. Further, a part or all of the processing functions of each of these devices may be achieved by cloud computing.

### Example of Hardware Configuration of Label Generation Device

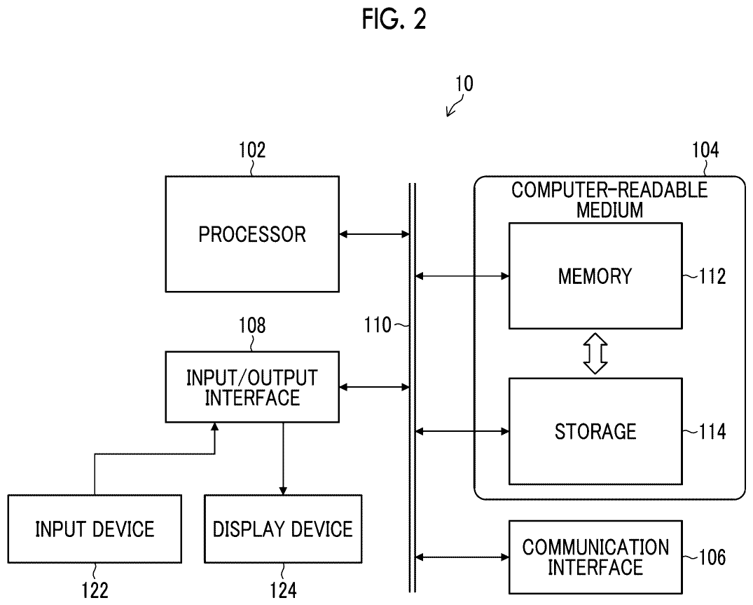

FIG. 2 is a block diagram showing an example of a hardware configuration of the label generation device 10 according to the embodiment. The label generation device 10 comprises a processor 102, a computer-readable medium 104 as a non-transitory tangible object, a communication interface 106, an input/output interface 108, and a bus 110. The processor 102 is connected to the computer-readable medium 104, the communication interface 106, and the input/output interface 108 via the bus 110.

A form of the label generation device 10 is not particularly limited, and may be a server, a workstation, a personal computer, and the like.

The processor 102 includes a central processing unit (CPU). The processor 102 may include a graphics processing unit (GPU). The processor 102 is an example of a “first processor” according to the present disclosure. The computer-readable medium 104 includes a memory 112 as a main storage device, and a storage 114 as an auxiliary storage device. The computer-readable medium 104 may be, for example, a semiconductor memory, a hard disk drive (HDD) device, a solid-state drive (SSD) device, or a combination of a plurality thereof. The computer-readable medium 104 is an example of a storage device that stores a command executed by the processor 102.

The label generation device 10 further comprises an input device 122 and a display device 124. The input device 122 is configured by, for example, a keyboard, a mouse, a multi-touch panel, other pointing devices, a voice input device, or an appropriate combination thereof.

The display device 124 is configured by, for example, a liquid crystal display, an organic electro-luminescence (OEL) display, a projector, or an appropriate combination thereof. The input device 122 and the display device 124 are connected to the processor 102 via the input/output interface 108. The label generation device 10 may be connected to the electric communication line 40 via the communication interface 106.

### Example 1 of Label Generation Method

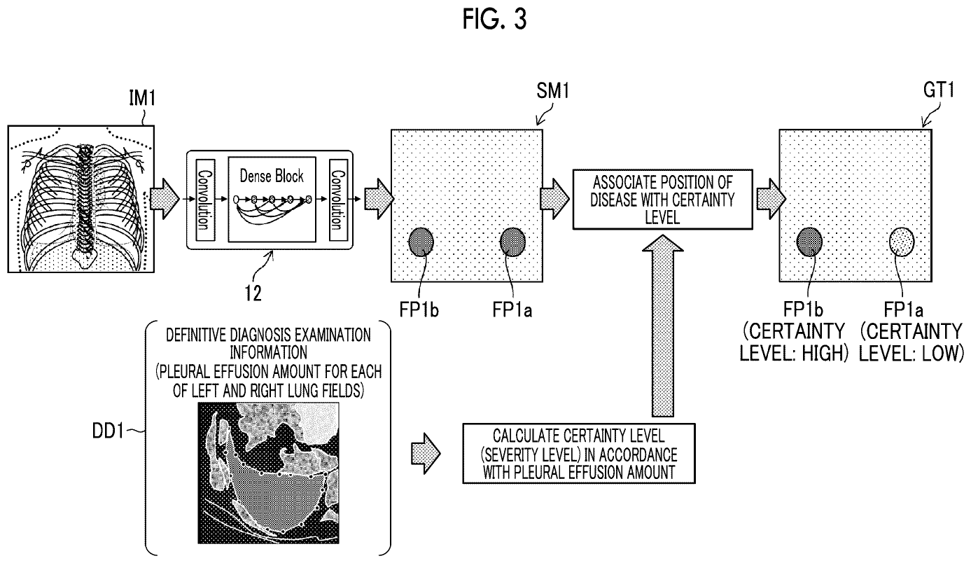

FIG. 3 is an explanatory diagram showing Example 1 of a label generation method executed by the label generation device 10. Here, a workflow will be described, which is for a case in which a medical image IM1 as an input image is a plain chest X-ray image, the definitive diagnosis examination is a CT examination, and the target disease is pleural effusion.

The processor 102 inputs the medical image IM1 to the first machine learning model 12 and acquires a saliency map SM1 indicating the candidate positions of the disease from the output of the first machine learning model 12. The medical image IM1 is an example of a “first medical image” and a “chest X-ray image” according to the present disclosure. The saliency map SM1 may be a binary image or a heat map image reflecting the probability of the disease (score indicating the likelihood of the disease).

A region FP1a and a region FP1b shown on the saliency map SM1 of FIG. 3 indicate positions of the finding (candidate positions of the disease) estimated by the first machine learning model 12. The region division granularity of the saliency map SM1, that is, the region division granularity of the candidate positions of the disease may be a unit of pixels of the medical image IM1. The unit of pixels, which is the region division granularity of the saliency map SM1, is an example of a “first division unit” according to the present disclosure.

In addition, the processor 102 acquires a pleural effusion amount for each of the left and right lung fields as definitive diagnosis examination information DD1. That is, the definitive diagnosis examination information DD1 includes the pleural effusion amount in the left lung field and the pleural effusion amount in the right lung field. It should be noted that the pleural effusion amount for each of the left and right lung fields can be measured, for example, from the CT image of the definitive diagnosis examination. The definitive diagnosis examination information DD1 may be data in a text format, such as a sentence, or may be data in a table format. The definitive diagnosis examination information DD1 is an example of “diagnostic information” according to the present disclosure.

The definitive diagnosis examination information DD1 includes information indicating the positions of the “left lung field” and the “right lung field”. The region division granularity of the position information included in the definitive diagnosis examination information DD1 uses, as a division unit, a part that is an anatomical structure such as the “left lung field” or the “right lung field”, and is coarser than the region division granularity of the saliency map SM1, and the region division granularity. The division unit of the position information included in the definitive diagnosis examination information DD1 is an example of a “second division unit” according to the present disclosure.

The processor 102 calculates a certainty level label (value indicating the certainty level) corresponding to the pleural effusion amount from the acquired definitive diagnosis examination information DD1. The pleural effusion amount is related to the severity level, and the degree (grade) of severity is higher as the value of the pleural effusion amount is larger. That is, a larger value is calculated as the value of the certainty level as the value of the pleural effusion amount is larger. For the method of calculating the certainty level from the pleural effusion amount, for example, a look-up table or a calculation expression may be used.

Here, for example, in a case in which a pleural effusion amount PEV_L in the left lung field is a value larger than a pleural effusion amount PEV_R in the right lung field, a certainty level label CL_L in the left lung field calculated from the pleural effusion amount PEV_L in the left lung field is a value larger than a certainty level label CL_R in the right lung field calculated from the pleural effusion amount PEV_R in the right lung field.

Subsequently, the processor 102 combines information on the candidate positions of the disease specified from the saliency map SM1 with the certainty level label calculated from the definitive diagnosis examination information DD1, to associate the candidate positions of the disease with the certainty level. In the example shown in FIG. 3, the certainty level label CL_L calculated from the pleural effusion amount PEV_L in the left lung field is associated with the position in the region FP1a belonging to the left lung field. In addition, the certainty level label CL_R calculated from the pleural effusion amount PEV_R in the right lung field is associated with the position in the region FP1b belonging to the right lung field.

The processor 102 may assign the certainty level label converted from the pleural effusion amount of the definitive diagnosis examination information DD1 as it is, as the certainty level of the disease at each position, to the candidate positions of the disease, or, for example, may assign the certainty level to each position by weighting the certainty level label in accordance with the values of the candidate positions of the saliency map SM1 (score values indicating the likelihood of the candidate positions of the disease).

In this way, the certainty level calculated based on the definitive diagnosis examination information DD1 is assigned to each of the candidate positions of the disease specified from the saliency map SM1, and the ground truth label of the position and the certainty level of the disease in the medical image IM1 is generated.

That is, in Example 1 of the label generation method according to the present embodiment, the disease position of the definitive diagnosis examination information DD1 is identified by using the disease saliency map SM1 obtained from the medical image IM1 as the prior knowledge, and the certainty level calculated from the definitive diagnosis examination information DD1 is used as the ground truth label.

Ground truth data GT1, which is data of the ground truth label generated for the medical image IM1, may be, for example, a gradation image obtained by gradation representation of the ground truth label assigned to each pixel of the medical image IM1. The ground truth data GT1 is data indicating the ground truth for the output with respect to the input of the medical image IM1 in supervised learning.

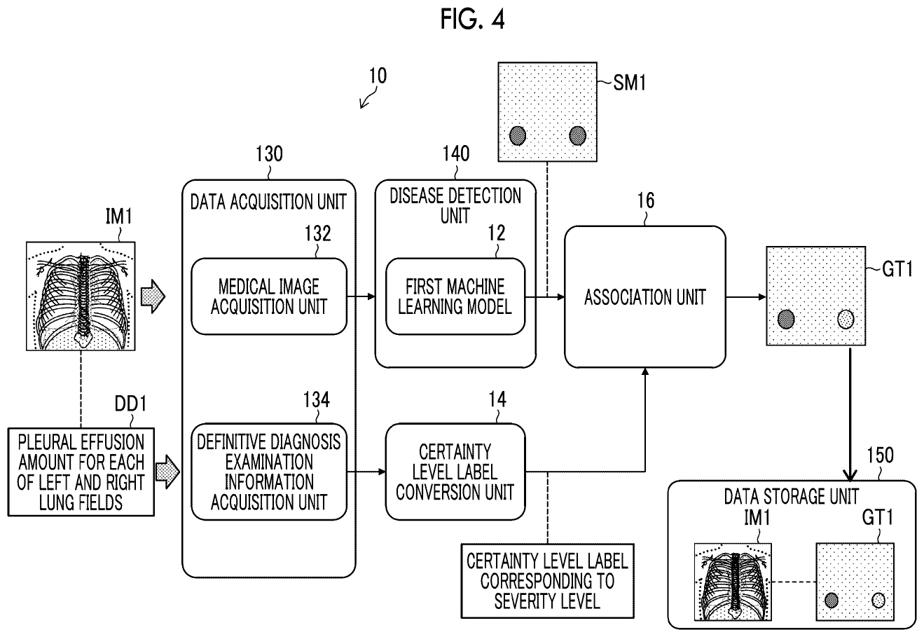

FIG. 4 is a block diagram schematically showing a functional configuration of the label generation device 10 that executes the label generation method shown in FIG. 3. The label generation device 10 comprises a data acquisition unit 130, a disease detection unit 140, a certainty level label conversion unit 14, an association unit 16, and a data storage unit 150.

The data acquisition unit 130 includes a medical image acquisition unit 132 and a definitive diagnosis examination information acquisition unit 134. The medical image acquisition unit 132 acquires the medical image IM1 as a processing target. The definitive diagnosis examination information acquisition unit 134 acquires data of the definitive diagnosis examination information DD1 that is linked with the medical image IM1. It should be noted that FIG. 4 shows an example in which the pleural effusion amount for each of the left and right lung fields is acquired as the definitive diagnosis examination information DD1.

The disease detection unit 140 includes the first machine learning model 12 and detects the position of the disease from the input medical image IM1. The saliency map SM1 indicating the candidate positions of the disease with respect to the medical image IM1 is obtained by the processing of the disease detection unit 140.

The certainty level label conversion unit 14 converts the definitive diagnosis examination information DD1 into the certainty level label corresponding to the severity level of the disease.

The association unit 16 generates the ground truth label of the position and the certainty level of the disease by associating the candidate positions of the disease specified from the saliency map SM1 with the certainty level calculated from the definitive diagnosis examination information DD1.

The association unit 16 may use the certainty level label converted from the definitive diagnosis examination information DD1 as it is as the ground truth label of the certainty level of each position for the candidate position of the disease, or may weight the certainty level label in accordance with the values (score values indicating the likelihood) of the candidate positions of the saliency map SM1 to determine the certainty level of each position.

The ground truth data GT1 generated by the association processing of the association unit 16 is stored in the data storage unit 150 in a state of being linked with the medical image IM1.

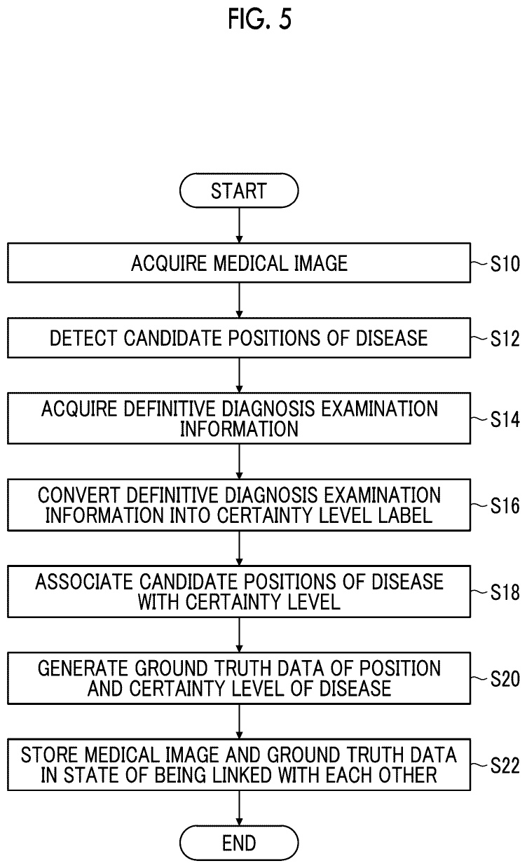

FIG. 5 is a flowchart showing Example 1 of the label generation method according to the embodiment. FIG. 5 is a flowchart of the explanatory diagram of FIG. 3.

In step S10, the processor 102 acquires the medical image IM1.

In step S12, the processor 102 detects the candidate positions of the disease in the acquired medical image IM1 by using the first machine learning model 12. In step S12, the processor 102 acquires the candidate positions of the disease as the detection results.

In step S14, the processor 102 acquires the definitive diagnosis examination information DD1 corresponding to the medical image IM1.

In step S16, the processor 102 converts the acquired definitive diagnosis examination information DD1 into the certainty level label.

It should be noted that the order of processing from step S10 to step S16 is not limited to the example shown in FIG. 5. For example, step S14 may be executed prior to step S10 or may be executed in parallel with step S10.

In step S18, the processor 102 associates the candidate positions of the disease with the certainty level. With this association, the processor 102 generates the ground truth data GT1 of the position and the certainty level of the disease with respect to the medical image IM1 (step S20).

In step S22, the processor 102 stores the medical image IM1 and the generated ground truth data GT1 in the data storage unit 150 in a state of being linked with each other.

After step S22, the processor 102 ends the flowchart of FIG. 5.

The processor 102 executes the processing of the flowchart of FIG. 5 on the paired data of the medical images IMi for the plurality of patients and the definitive diagnosis examination information DDi, so that the ground truth data GTi is generated for each of a plurality of medical images IMi, and the data set DS including a plurality of sets of the pair of the medical image IMi and the ground truth data GTi is obtained. It should be noted that the subscript i is an index number for identifying the paired data.

### Example 2 of Label Generation Method

FIG. 3 shows an example in which the ground truth label of the position and the certainty level of the disease is generated in the region division granularity (hereinafter, referred to as first granularity) of the saliency map SM1, but the present invention is not limited to this example, and the ground truth label of the position and the certainty level of the disease may be generated in the region division granularity (hereinafter, referred to as second granularity) of the position of the disease in the definitive diagnosis examination information DD1.

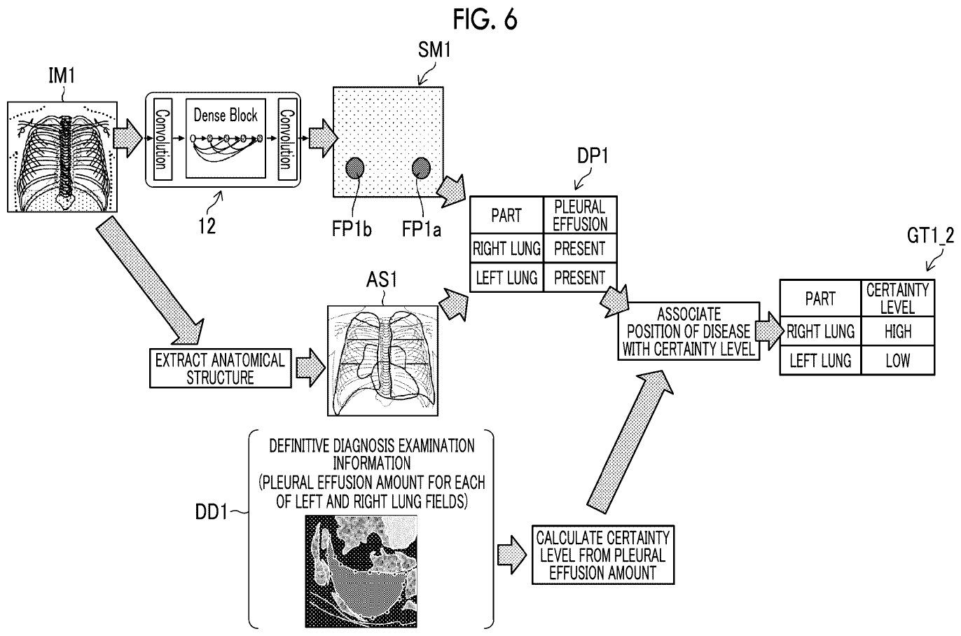

FIG. 6 is an explanatory diagram showing Example 2 of the label generation method executed by the label generation device 10. A difference of FIG. 6 from FIG. 3 will be described.

In FIG. 6, the processor 102 performs processing of extracting a region of an anatomical structure from the input medical image IM1, to acquire anatomical structure information AS1. In a case in which the target disease is pleural effusion, the processor 102 may extract the region (part) of each of the left lung field and the right lung field in the medical image IM1, and acquire the anatomical structure information AS1 in which the regions of the left and right lung fields are specified. The region division granularity of the anatomical structure information AS1 shown in the example of FIG. 5 may be the same granularity (second granularity) as the definitive diagnosis examination information DD1.

The processor 102 further combines the saliency map SM1 and the anatomical structure information AS1 to generate disease position data DP1 indicating the position of the disease in the second granularity. The disease position data DP1 shown in FIG. 6 may be table data indicating that there is pleural effusion in each of the right lung and the left lung.

The processor 102 associates each position (here, each part) specified by the disease position data DP1 with the certainty level label converted from the definitive diagnosis examination information DD1, and generates ground truth data GT1_2 of the position and the certainty level of the disease.

In FIG. 6, a label with a high certainty level is assigned to the position of the right lung, and a label with a low certainty level is assigned to the position of the left lung. This is an example of a case in which the certainty level label of each position is generated in the second granularity. The ground truth data GT1_2 may be data in a table format.

### Example 3 of Label Generation Method

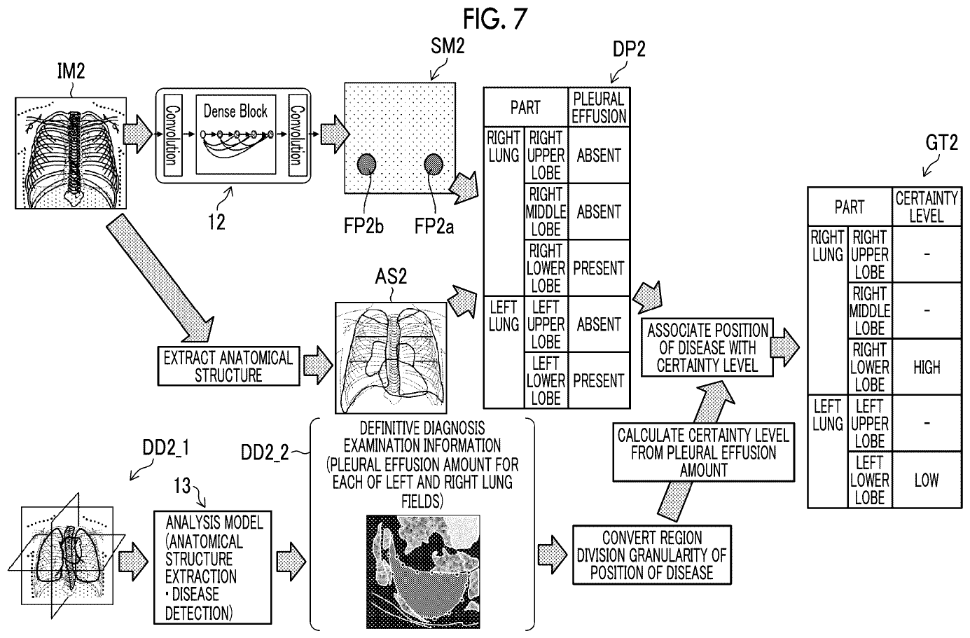

FIG. 6 shows an example in which the ground truth label of the position and the certainty level of the disease is generated in the region division granularity (second granularity) of the definitive diagnosis examination information DD1, but the present invention is not limited to this example, and the ground truth label of the position and the certainty level of the disease may be generated in third region division granularity (hereinafter, referred to as third granularity) different from both the region division granularity (first granularity) of the saliency map SM1 and the region division granularity (second granularity) of the position of the disease in the definitive diagnosis examination information DD1. The example thereof is shown in FIG. 7.

FIG. 7 is an explanatory diagram showing Example 3 of the label generation method executed by the label generation device 10. A difference of FIG. 7 from FIG. 6 will be described.

In FIG. 7, a combination of a medical image IM2 and definitive diagnosis examination information DD2_1 is used instead of a combination of the medical image IM1 and the definitive diagnosis examination information DD1 in FIG. 6.

The medical image IM2 may be a plain X-ray image as in the medical image IM1. The definitive diagnosis examination information DD2_1 may be a CT image (computed tomography image) which is a three-dimensional examination image obtained by the CT examination as the definitive diagnosis examination.

The processor 102 generates a saliency map SM2 from the input medical image IM2 by the first machine learning model 12. Further, the processor 102 extracts the anatomical structure from the medical image IM2 to acquire anatomical structure information AS2.

The processor 102 may acquire the anatomical structure information AS2 by using a machine learning model that has been trained (subjected to learning) through the machine learning so as to perform a segmentation task of recognizing the anatomical structure in units of pixels from the input medical image IM2 and performing labeling in accordance with the classification of the region of the anatomical structure.

In the example shown in FIG. 7, a segmentation image is obtained by extracting the region in units of pixels from the plain chest X-ray image, recognizing the anatomical structure such as the clavicle, the trachea, the right lung field, the left lung field, the superior vena cava, the right atrium, the great vessel arch, the descending aorta, and the left atrium, and labeling the type of the anatomical structure in units of pixels. There may be various aspects for the type of the anatomical structure to be extracted. For example, the lung may be classified into units of sections such as a right upper lobe, a right middle lobe, a right lower lobe, a left upper lobe, and a left lower lobe, which are further subdivided from the classification such as the right lung field and the left lung field.

Here, in order to show an example of the region division granularity different from the region division granularity (second granularity) of the disease position specified by the definitive diagnosis examination, it is assumed that the anatomical structure information AS2 includes the position information in units of five classifications of sections finer than the two classifications such as the left lung field and the right lung field for the lung. It should be noted that the anatomical structure information AS2 may information for specifying the position of the region division granularity based on the anatomical structure such as the left lung field and the right lung field.

The processor 102 generates disease position data DP2 indicating the candidate positions of the disease having the region division granularity different from the saliency map SM2 by combining the saliency map SM2 and the anatomical structure information AS2. For example, the disease position data DP2 may be data in a table format as shown in FIG. 7. The division unit (region division granularity) of the position information included in the disease position data DP2 is an example of a “third division unit” according to the present disclosure.

In addition, the processor 102 analyzes the CT image by using the analysis model 13, extracts the anatomical structure, and detects the disease from the CTimage. The analysis model 13 may be a trained machine learning model that has been trained in advance through the machine learning so as to perform the labeling of the anatomical structure and the detection of the disease in units of voxels from the input CT image. The analysis model 13 may be a combination of a model that extracts the anatomical structure and a model that detects the disease. As a machine learning model that executes three-dimensional segmentation, for example, a neural network model using a three-dimensional U-Net architecture can be applied.

The analysis model 13 according to the present example extracts, for example, the left lung field and the right lung field as the anatomical structures from the CT image. Further, the analysis model 13 detects a pleural effusion region from the CT image.

The processor 102 calculates the pleural effusion amount for each of the left and right lung fields based on an analysis result of the analysis model 13. The value of the pleural effusion amount for each of the left and right lung fields calculated in this way corresponds to the definitive diagnosis examination information DD1 shown in FIG. 6.

The pleural effusion amount for each of the left and right lung fields calculated based on the definitive diagnosis examination information DD2_1 shown in FIG. 7 is understood as definitive diagnosis examination information DD2_2 that is potentially inherent in the definitive diagnosis examination information DD2_1. Stated another way, it is understood that the definitive diagnosis examination information DD2_1, which is the three-dimensional examination image, includes information for specifying the position of the pleural effusion in units of voxels and information for specifying the position of the pleural effusion in units of anatomical structures.

The processor 102 converts the pleural effusion amount for each of the left and right lung fields calculated from the CT image into the certainty level label. As a result, data of the certainty level label reflecting the severity level of the pleural effusion in each of the left lung field and the right lung field is obtained. The granularity of the position information for specifying the position of the pleural effusion in the label data of the certainty level is different from the granularity of the position information in the disease position data DP2.

In the example of FIG. 7, a magnitude relationship among the first granularity, which is the region division granularity of the saliency map SM2, the second granularity, which is the region division granularity of the position of the disease (here, the position of the pleural effusion) in the label data of the certainty level for each of the left and right lung fields, and the third granularity, which is the region division granularity of the disease position data DP2, is first granularity<third granularity<second granularity.

The processor 102 converts the label data of the certainty level into data in the third granularity in order to match the granularity of the position information in the disease position data DP2 and the granularity of the position information in the label data of the certainty level.

Then, the processor 102 combines the label data of the certainty level converted into the third granularity with the disease position data DP2 to associate each candidate position of the disease position data DP2 with the certainty level, and generates ground truth data GT2 of the position and the certainty level of the disease with respect to the medical image IM2. In this way, the ground truth data GT is obtained in which the position and the certainty level of the disease are specified in the third granularity.

It should be noted that, in the ground truth data GT2 shown in FIG. 7, “high” in the certainty level indicates that a numerical value indicating a relatively high certainty level is assigned, and “low” in the certainty level indicates that a numerical value indicating a relatively low certainty level is assigned. For example, in a case in which the certainty level corresponding to the severity level of the disease is represented by a numerical value in a range of 0 to 1, “high” may be “1” and “low” may be “0.2”. In addition, “-” of the certainty level for the non-disease position of the ground truth data GT2 shown in FIG. 7 may be “0”.

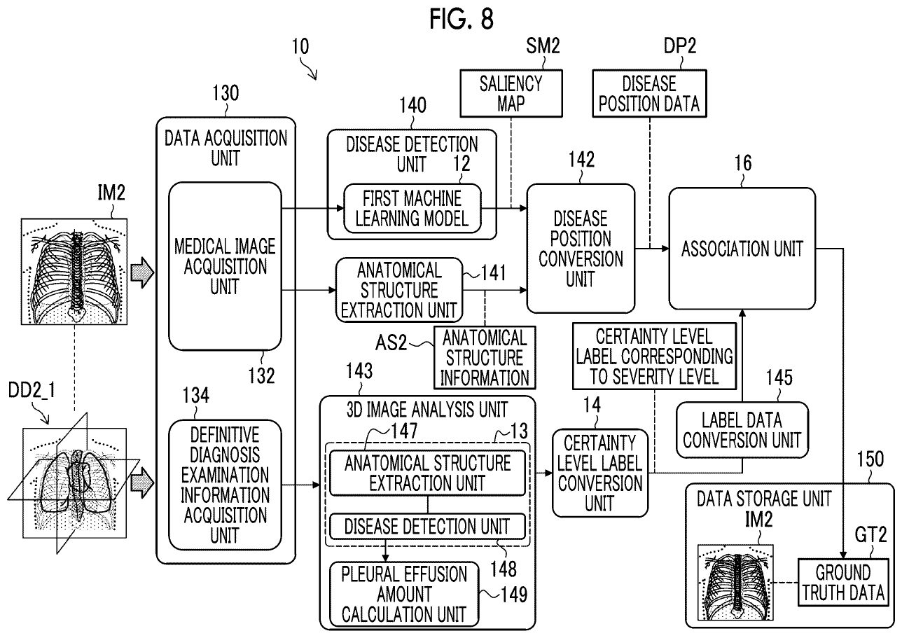

FIG. 8 is a functional block diagram of the label generation device 10 that executes the label generation method shown in FIG. 7. A difference of the configuration shown in FIG. 8 from FIG. 4 will be described. The label generation device 10 shown in FIG. 8 further includes an anatomical structure extraction unit 141, a disease position conversion unit 142, a 3D image analysis unit 143, and a label data conversion unit 145, in addition to the configuration shown in FIG. 4. It should be noted that the notation “3D” means “three-dimensional”.

The anatomical structure extraction unit 141 extracts the anatomical structure from the medical image IM2 acquired via the medical image acquisition unit 132, and acquires the anatomical structure information AS2.

The disease position conversion unit 142 converts the information on the candidate positions of the disease shown in the saliency map SM2 into the information on the candidate positions having different region division granularity. The disease position conversion unit 142 generates the disease position data DP2 by converting, for example, information on the candidate positions in units of pixels in the medical image IM2 into information on the candidate positions in the region division granularity of the anatomical structure indicated in the anatomical structure information AS2.

The 3D image analysis unit 143 includes the analysis model 13 that analyzes the CT image which is the three-dimensional examination image acquired via the definitive diagnosis examination information acquisition unit 134. The analysis model 13 functions as an anatomical structure extraction unit 147 that extracts the anatomical structure from the CT image and a disease detection unit 148 that detects the disease from the CT image. The disease detection unit 148 detects the pleural effusion region from, for example, the CT image.

In addition, the 3D image analysis unit 143 includes a pleural effusion amount calculation unit 149, and the pleural effusion amount calculation unit 149 counts voxels of the pleural effusion region in the CT image based on the detection result of the disease detection unit 148, to calculate the pleural effusion amount for each of the left and right lung fields. The pleural effusion amount calculation unit 149 may calculate the pleural effusion amount based on information on the pleural effusion region designated on the CT image via a user interface.

The certainty level label conversion unit 14 converts the pleural effusion amount for each of the left and right lung fields acquired by the analysis via the 3D image analysis unit 143 into the certainty level label.

The label data conversion unit 145 converts the certainty level label for each of the left and right lung fields into the label data having the same region division granularity as the disease position data DP2. Here, the label data in the second granularity is converted into the label data in the third granularity.

The association unit 16 combines the disease position data DP2 and the certainty level label acquired from the definitive diagnosis examination information DD2 by the label data conversion unit 145, associates the certainty level label with each candidate position of the disease in the disease position data DP2, and generates the ground truth label of the position and the certainty level of the disease. The ground truth data GT2 generated by the association unit 16 is stored in the data storage unit 150 in a state of being linked with the medical image IM2.

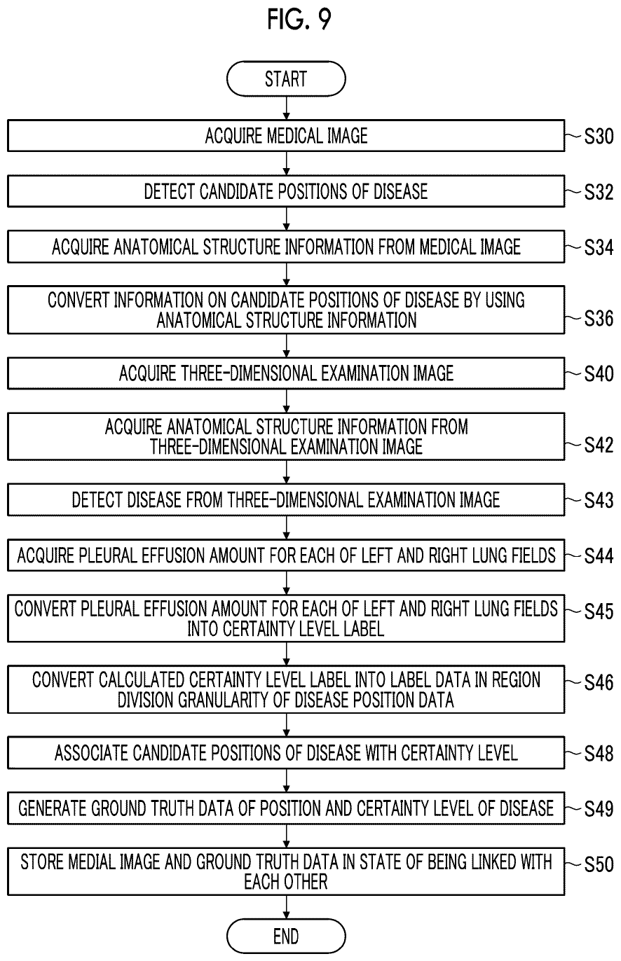

FIG. 9 is a flowchart showing Example 3 of the label generation method according to the embodiment. FIG. 9 is a flowchart of the explanatory diagram of FIG. 8.

In step S30, the processor 102 acquires the medical image IM2.

In step S32, the processor 102 detects the candidate positions of the disease in the acquired medical image IM2 by using the first machine learning model 12. In step S12, the processor 102 acquires the candidate positions of the disease as the detection results. That is, the processor 102 acquires the saliency map SM2 indicating candidate positions of the disease with respect to the medical image IM2.

In step S34, the processor 102 extracts the anatomical structure from the medical image IM2, and acquires the anatomical structure information AS2.

In step S36, the processor 102 converts the information on the candidate positions of the disease shown in the saliency map SM2 into the disease position data DP2 in the anatomical structure unit.

In addition, the processor 102 may impose a constraint such that the candidate positions of the disease in the saliency map SM2 are located within a desired anatomical structure, by using the anatomical structure information AS2. For example, the processor 102 may exclude, from the candidate positions, the candidate positions located outside the lung region including the left lung field and the right lung field among the candidate positions of the pleural effusion estimated by the first machine learning model 12, and may use only the candidate positions located in the lung region as the appropriate candidate positions. Since it is assumed that the first machine learning model 12 may output an erroneous candidate position depending on the inference performance of the first machine learning model 12, it is desirable to impose a constraint such that the position of the disease is located within a desired anatomical structure, by using the anatomical structure information AS2 in combination.

In step S40, the processor 102 acquires the three-dimensional examination image of the definitive diagnosis examination with respect to the medical image IM2. The three-dimensional examination image is, for example, the CT image.

In step S42, the processor 102 extracts the anatomical structure from the three-dimensional examination image, to acquire the anatomical structure information.

In step S43, the processor 102 detects the disease from the three-dimensional examination image. The disease as a detection target is, for example, pleural effusion, and the processor 102 extracts the pleural effusion region from the three-dimensional examination image.

In step S44, the processor 102 calculates the pleural effusion amount based on the detection result in step S43, to acquire the pleural effusion amount for each of the left and right lung fields.

In step S45, the processor 102 converts the pleural effusion amount for each of the left and right lung fields into the certainty level.

In step S46, the certainty level label for each of the left and right lung fields obtained in step S45 is converted into the label data having the same region division granularity as the disease position data DP2.

It should be noted that the order of processing from step S30 to step S46 is not limited to the example in FIG. 9, and the order can be changed as long as no contradiction occurs in the processing. For example, step S40 may be executed prior to step S30 or may be executed in parallel with step S30.

In step S48, the processor 102 associates the candidate positions of the disease with the certainty level. With this association, the processor 102 generates the ground truth data GT2 of the position and the certainty level of the disease with respect to the medical image IM2 (step S49).

In step S50, the processor 102 stores the medical image IM2 and the generated ground truth data GT2 in the data storage unit 150 in a state of being linked with each other.

After step S50, the processor 102 ends the flowchart of FIG. 9.

The flowchart shown in FIG. 9 is repeatedly executed on the paired data of the medical images for the plurality of patients and the three-dimensional examination images of the definitive diagnosis examination. By the processor 102 executing the processing of the flowchart of FIG. 9 on the plurality of pieces of the paired data, the ground truth data GTi is generated for each of the plurality of medical images IMi, and the data set DS including a plurality of sets of the pair of the medical image IMi and the ground truth data GTi is obtained.

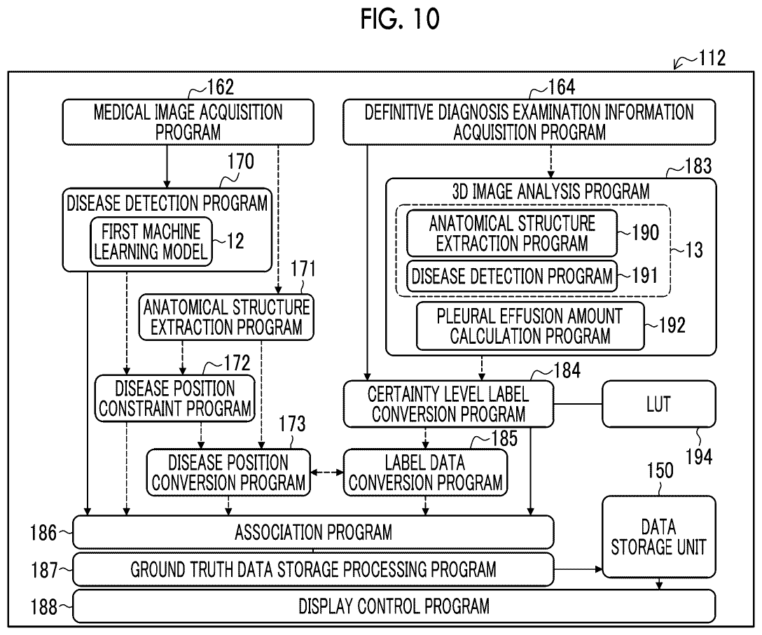

FIG. 10 is a block diagram showing an example of the program and the data stored in the memory 112 of the label generation device 10 that executes the label generation method of the flowchart shown in FIG. 9.

A plurality of programs including a medical image acquisition program 162, a definitive diagnosis examination information acquisition program 164, a disease detection program 170, an anatomical structure extraction program 171, a disease position constraint program 172, a disease position conversion program 173, a 3D image analysis program 183, a certainty level label conversion program 184, a label data conversion program 185, an association program 186, a ground truth data storage processing program 187, and a display control program 188, and the data are stored in the memory 112. The term “program” includes the concept of a program module. The processor 102 (see FIG. 2) functions as various processing units by executing the commands of the program stored in the memory 112.

The medical image acquisition program 162 includes a command to execute processing of acquiring the medical image, and achieves the function as the medical image acquisition unit 132.

The definitive diagnosis examination information acquisition program 164 includes a command to execute processing of acquiring the definitive diagnosis examination information, and achieves the function as the definitive diagnosis examination information acquisition unit 134. The disease detection program 170 includes the first machine learning model 12. The disease detection program 170 includes a command to execute processing of detecting the disease from the input medical image, and achieves the function as the disease detection unit 140.

The anatomical structure extraction program 171 includes a command to execute processing of recognizing the anatomical structure from the medical image and generating the anatomical structure information, and achieves the function as the anatomical structure extraction unit 141.

The disease position constraint program 172 includes a command to execute processing of constraining the position of the disease within the region of the anatomical structure by using the candidate position of the disease detected by the disease detection program 170, and the anatomical structure information generated by the anatomical structure extraction program 171.

The disease position conversion program 173 includes a command to execute processing of converting the information on the candidate position of the disease detected by the disease detection program 170 into the position information having desired region division granularity. For example, the disease position conversion program 173 achieves a processing function of converting the information on the candidate positions of the disease specified in units of pixels of the medical image into the disease position data DP2 indicating the candidate positions of the disease in units of regions of the anatomical structure.

The 3D image analysis program 183 is a program for executing processing of analyzing the three-dimensional examination image, and includes an anatomical structure extraction program 190, a disease detection program 191, and a pleural effusion amount calculation program 192. The anatomical structure extraction program 190 includes a command to execute processing of extracting the anatomical structure from the three-dimensional examination image and performing the labeling in accordance with the classification of the anatomical structure. The anatomical structure extraction program 190 extracts, for example, the regions of the left lung field and the right lung field from the CT image.

The disease detection program 191 includes a command to execute processing of detecting the disease from the three-dimensional examination image. The disease detection program 191 includes, for example, a command to execute processing of detecting the pleural effusion region from the CT image. It should be noted that the anatomical structure extraction program 190 and the disease detection program 191 may be configured as the analysis model 13.

The pleural effusion amount calculation program 192 includes a command to execute processing of calculating the pleural effusion amount from the pleural effusion region in the three-dimensional examination image.

The certainty level label conversion program 184 includes a command to execute processing of converting the pleural effusion amount into the certainty level label. The certainty level label conversion program 184 is configured to perform the conversion processing by using, for example, a look-up table 194 that describes a correspondence relationship between the pleural effusion amount and the certainty level.

The label data conversion program 185 includes a command to execute processing of converting data of the certainty level label (in the second granularity) for each of the left and right lung fields obtained from the definitive diagnosis examination information into label data having the same granularity (third granularity) as the disease position data generated by the disease position conversion program 173.

The association program 186 includes a command to execute processing of combining the disease position data DP2 obtained from the medical image with the label data of the certainty level obtained from the definitive diagnosis examination information DD2, associating the position of the disease with the certainty level, and generating the ground truth label of the position and the certainty level of the disease. The association program 186 achieves the function of the association unit 16.

The ground truth data storage processing program 187 includes a command to execute processing of storing, in the data storage unit 150, the ground truth data generated by the association program 186 in a state of being linked with the medical image. A storage area as the data storage unit 150 may be provided in the storage 114.

The display control program 188 includes a command to generate a display signal required for display output to the display device 124 and execute display control of the display device 124.

### Example of Using Sputum Examination Information as Definitive Diagnosis Examination Information

In a case in which the sputum examination information is used as the definitive diagnosis examination information, the processor 102 calculates the certainty level label of the disease based on the amount of bacteria collected in the sputum examination in a case in which the sputum examination information is converted into the certainty level label. Other processing may be the same as the above-described processing in the label generation device 10.

### Example of Machine Learning Device

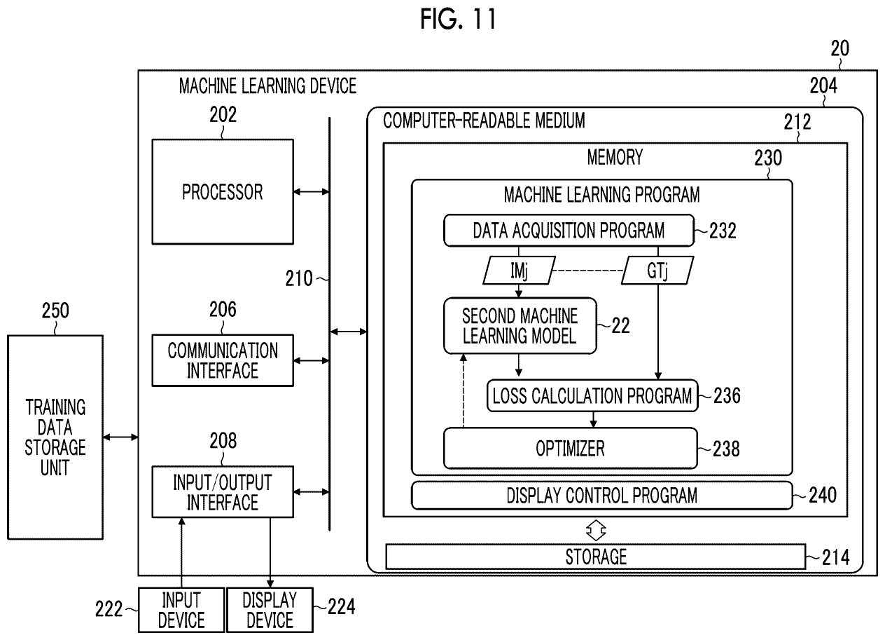

FIG. 11 is a block diagram showing an example of a hardware configuration of the machine learning device 20 according to the embodiment. The machine learning device 20 comprises a processor 202, a computer-readable medium 204, which is a non-transitory tangible object, a communication interface 206, an input/output interface 208, and a bus 210. The computer-readable medium 204 includes a memory 212 and a storage 214. The processor 202 is connected to the computer-readable medium 204, the communication interface 206, and the input/output interface 208 via the bus 210. The machine learning device 20 may further comprise an input device 222 and a display device 224. The hardware configuration of the machine learning device 20 may be the same as the corresponding components of the label generation device 10 shown in FIG. 2. The processor 202 is an example of a “second processor” according to the present disclosure.

A form of the machine learning device 20 is not particularly limited, and may be a server, a workstation, a personal computer, and the like.

The machine learning device 20 is communicably connected to an external device, such as a training data storage unit 250, via the communication interface 206. The training data storage unit 250 includes a storage in which a training data set including a plurality of training data is stored. It should be noted that the training data storage unit 250 may be constructed in the storage 214 in the machine learning device 20.

The computer-readable medium 204 stores various programs, including a machine learning program 230 and a display control program 240, along with data.

The machine learning program 230 includes a command to acquire the training data and execute learning processing of the second machine learning model 22. That is, the machine learning program 230 includes a data acquisition program 232, a second machine learning model 22, a loss calculation program 236, and an optimizer 238.

The data acquisition program 232 includes a command to execute processing of acquiring the training data in which a medical image IMj and ground truth data GTj are linked with each other from the training data storage unit 250.

The second machine learning model 22 receives the input of the medical image IMj, estimates the position and the certainty level of the disease from the input medical image IMj, and outputs an estimation result. The medical image IMj is an example of a “second medical image” according to the present disclosure.

The loss calculation program 236 includes a command to execute processing of calculating a loss indicating an error between the output data of the second machine learning model 22 and the ground truth data GTj. The optimizer 238 includes a command to execute processing of calculating an update amount of the parameters of the second machine learning model 22 from the calculated loss and updating the parameters of the second machine learning model 22 based on the calculated update amount.

The display control program 240 includes a command to generate a display signal required for display output to the display device 224 and execute display control of the display device 224.

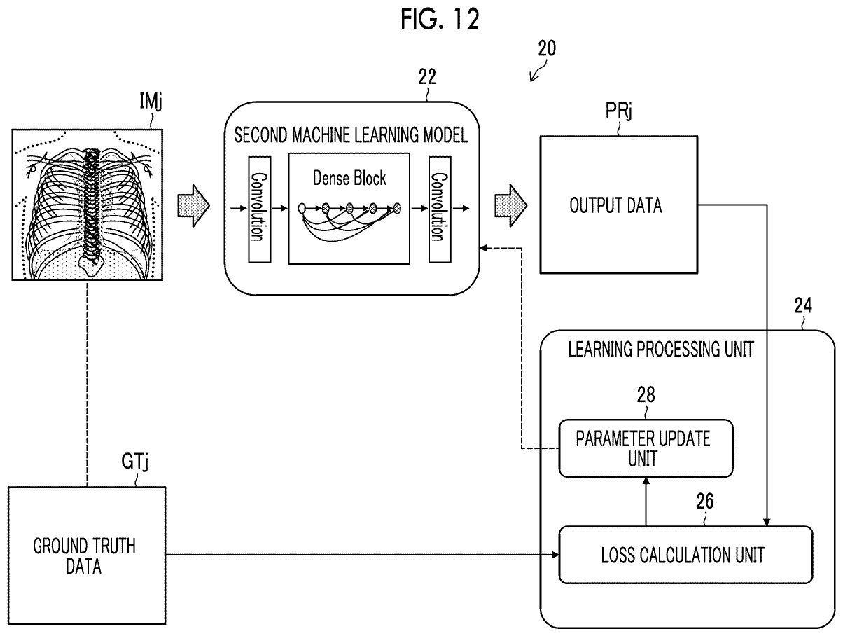

FIG. 12 is a block diagram schematically showing a functional configuration of the machine learning device 20. The machine learning device 20 includes the second machine learning model 22 and a learning processing unit 24. The learning processing unit 24 includes a loss calculation unit 26 and a parameter update unit 28.

The loss calculation unit 26 calculates a loss indicating an error between output data PRj indicating the position and the certainty level of the disease output from the second machine learning model 22 and the ground truth data GTj linked with the medical image IMj.

The parameter update unit 28 calculates an update amount of the parameters of the second machine learning model 22 such that the loss is decreased, that is, the output data PRj is close to the ground truth data GTj, based on the loss calculated by the loss calculation unit 26, and updates the parameters of the second machine learning model 22 in accordance with the calculated update amount. The parameters of the second machine learning model 22 include, for example, filter coefficients (weights of connections between nodes) of filters used for processing of each layer of a neural network, biases of the nodes, and the like. The parameter update unit 28 optimizes the parameters of the model by using, for example, a method such as a stochastic gradient descent (SGD) method.

The learning processing is performed using the plurality of training data, and the update of the parameters of the second machine learning model 22 is repeated, so that the parameters of the second machine learning model 22 are optimized, and the second machine learning model 22 is trained to output an estimation result similar to the ground truth data GTj with respect to the input of the medical image IMj.

In a case in which the certainty level of the disease is represented by a continuous value, the second machine learning model 22 may be configured as a regression model that performs regression-prediction of the certainty level of the disease from the input medical image IMj.

In addition, in a case in which the certainty level of the disease is represented by a discrete value, the second machine learning model 22 may be configured as a classification model that performs classification-prediction of the certainty level of the disease from the input medical image IMj.

### Example of Trained Model Generation Method

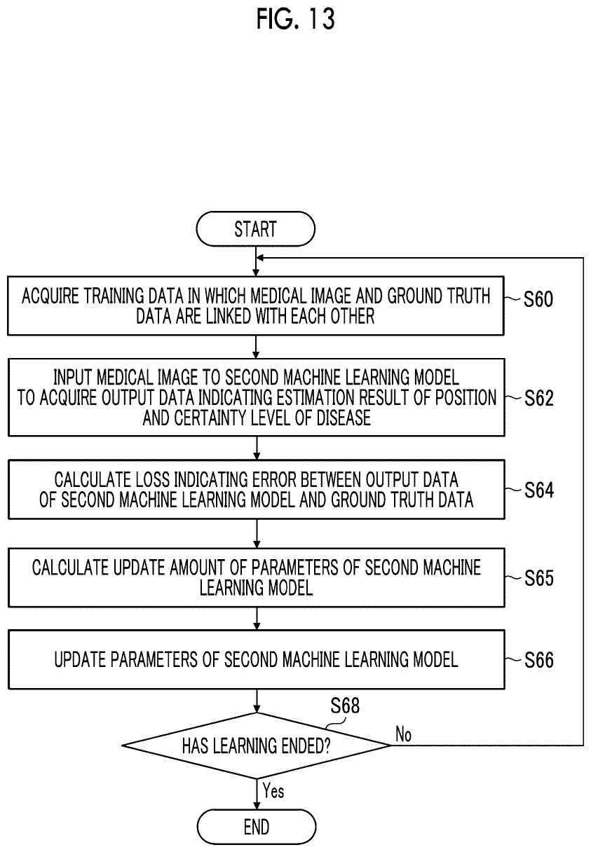

FIG. 13 is a flowchart showing an example of a machine learning method executed by the machine learning device 20.

In step S60, the processor 202 acquires the training data which is a data pair in which the medical image IMj and the ground truth data GTj are linked with each other, from the training data set.

In step S62, the processor 202 inputs the acquired medical image IMj to the second machine learning model 22 to acquire the output data PRj indicating the estimation result of the position and the certainty level of the disease in the medical image IMj from the second machine learning model 22. For example, the second machine learning model 22 performs regression- prediction of the certainty level of the disease from the medical image IMj and outputs the prediction result (estimation result). Alternatively, the second machine learning model 22 performs classification-prediction of the certainty level of the disease from the medical image IMj and outputs the prediction result.

In step S64, the processor 202 calculates the loss indicating the error between the output data PRj of the second machine learning model 22 and the ground truth data GTj.

In step S65, the processor 202 calculates the update amount of the parameters of the second machine learning model 22 such that the loss calculated in step S64 is decreased.

In step S66, the processor 202 updates the parameters of the second machine learning model 22 in accordance with the update amount of the parameters calculated in step S65. The above-described operations of step S60 to Step S66 may be performed in units of mini-batches.

After step S66, in step S68, the processor 202 determines whether or not to end the learning. A learning end condition may be determined based on the value of the loss, or may be determined based on the number of updates of the parameters. As for a method based on the value of the loss, for example, the learning end condition may include the loss converging within a prescribed range. Also, as for a method based on the number of updates, for example, the learning end condition may include that the number of updates reaches a predetermined number of times. Alternatively, a data set for performance evaluation of the model may be prepared separately from the training data set, and whether or not to end the learning may be determined based on an evaluation value obtained by using the evaluation data.

In a case in which a No determination is made as a determination result in step S68, the processor 202 returns to step S60 and continues the learning processing. On the other hand, in a case in which a Yes determination is made as the determination result in step S68, the processor 202 terminates the flowchart of FIG. 13.

In this way, the generated second machine learning model 22 that has trained (subjected to learning) is a disease detection model that receives the input of the unknown medical image IMu and outputs the position and the certainty level of the disease with respect to the medical image IMu. The machine learning method executed by the machine learning device 20 can be understood as a method of generating the trained second machine learning model 22, and is an example of a “trained model generation method” according to the present disclosure.

### Example of Image Processing Device

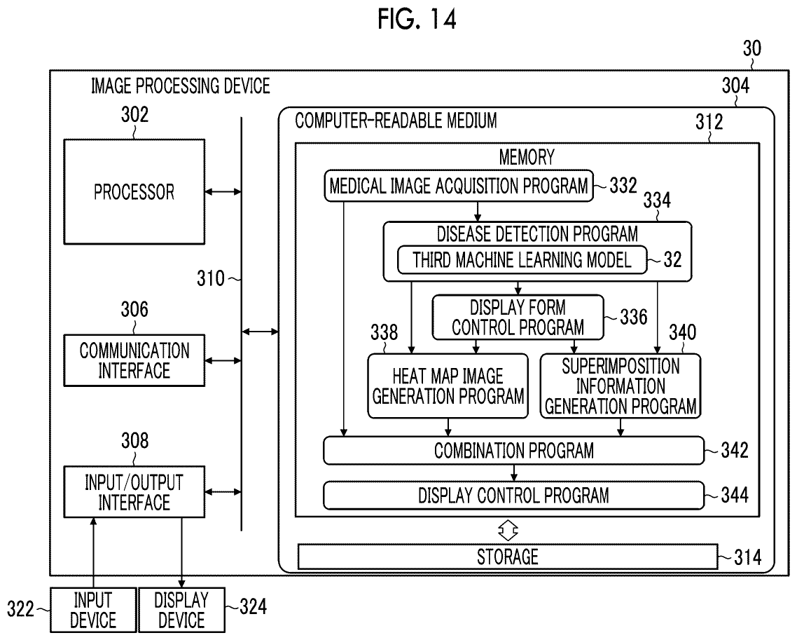

FIG. 14 is a block diagram showing an example of a hardware configuration of the image processing device 30 according to the embodiment. The image processing device 30 comprises a processor 302, a computer-readable medium 304 as a non-transitory tangible object, a communication interface 306, an input/output interface 308, and a bus 310. The computer-readable medium 304 includes a memory 312 and a storage 314. The processor 302 is connected to the computer-readable medium 304, the communication interface 306, and the input/output interface 308 via the bus 310. The image processing device 30 may further comprise an input device 322 and a display device 324. The hardware configuration of the image processing device 30 may be the same as the corresponding components of the label generation device 10 shown in FIG. 2. The processor 302 is an example of a “third processor” according to the present disclosure.

A form of the image processing device 30 is not particularly limited, and may be a server, a workstation, a personal computer, and the like.

The computer-readable medium 304 stores various programs including a medical image acquisition program 332, a disease detection program 334, a display form control program 336, a heat map image generation program 338, a superimposition information generation program 340, a combination program 342, and a display control program 344, along with data.

The medical image acquisition program 332 includes a command to execute processing of acquiring the medical image IMu as a processing target.

The disease detection program 334 includes a third machine learning model 32 that has been trained in advance. The disease detection program 334 includes a command to execute processing of inferring the position and the certainty level of the disease from the medical image IMu.

The display form control program 336 includes a command to execute processing of controlling a display form in a case of displaying the detection result of the disease obtained from the disease detection program 334.

The heat map image generation program 338 includes a command to execute processing of generating a heat map image showing the position and the certainty level of the disease based on the detection result of the disease obtained from the disease detection program 334. The heat map image represents a distribution of the positions and the certainty levels of the disease. The heat map image displays a color in a changed manner in accordance with a value of the certainty level. For example, the heat map image represents the distribution of the values of the certainty level by changing the color in an order of red, orange, yellow, green, blue, indigo, and violet from the highest certainty level.

The superimposition information generation program 340 includes a command to execute processing of generating superimposition information for the medical image IMu based on the detection result of the disease detection program 334. The superimposition information is information on the disease detected from the medical image IMu, and may be, for example, a character, a symbol, or a figure, or a combination thereof. The superimposition information may include, for example, a character string that specifies a type of the disease (disease name), a symbol or a character string that indicates grade classification of the severity level of the disease, a rectangular frame that indicates the position of the disease, a numerical value that indicates a size of the disease region, and the like.

The combination program 342 includes a command to execute processing of generating a composite image in which the heat map image and the superimposition information are superimposed on the medical image IMu.

The display control program 344 includes a command to generate a display signal required for display output to the display device 324 and execute display control of the display device 324.

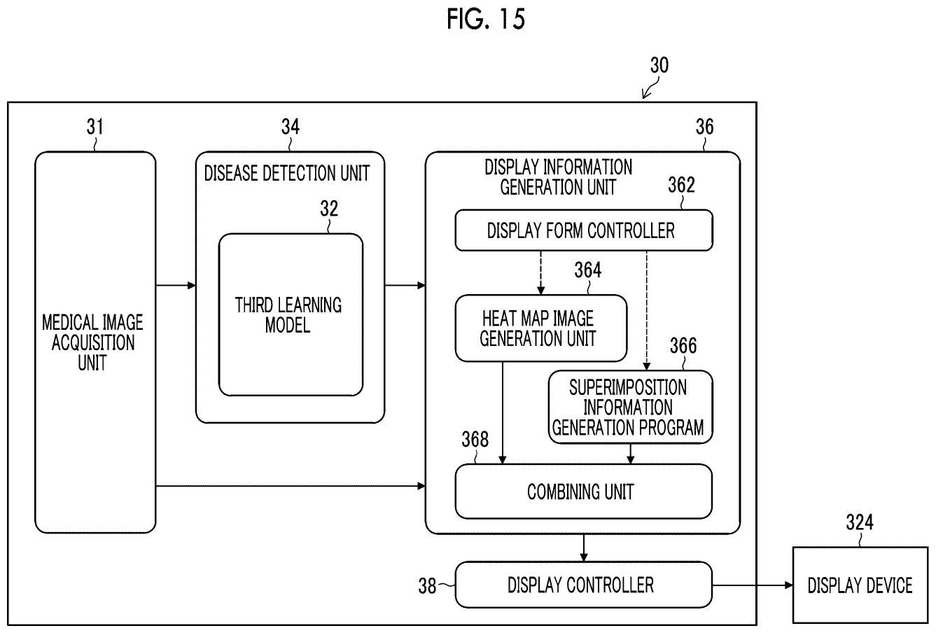

FIG. 15 is a block diagram schematically showing a functional configuration of the image processing device 30. The image processing device 30 includes a medical image acquisition unit 31, a disease detection unit 34, a display information generation unit 36, and a display controller 38. The medical image acquisition unit 31 acquires the medical image IMu. The disease detection unit 34 includes the third machine learning model 32, receives the input of the medical image IMu, and outputs the estimation result of the position and the certainty level of the disease from the medical image IMu.

The display information generation unit 36 is a processing unit that generates display information for displaying, in a visible manner, the result obtained by the inference using the third machine learning model 32. The display information generation unit 36 includes a display form controller 362, a heat map image generation unit 364, a superimposition information generation unit 366, and a combining unit 368.

The display form controller 362 controls a display form of information in a case of presenting the detection result of the disease detection unit 34 in accordance with the certainty level of the disease output from the third machine learning model 32. The display form controller 362 may control the display form by changing the processing of visualizing at least one of the heat map image or the superimposition information. For example, the display form controller 362 may display the heat map image in a color that is changed in accordance with the certainty level. In addition, the display form controller 362 may display an alert in a case in which the disease with a high certainty level is detected.

The heat map image generation unit 364 generates the heat map image showing the position and the certainty level of the disease based on the output of the third machine learning model 32 and the control from the display form controller 362.

The superimposition information generation unit 366 generates the superimposition information based on the output of the third machine learning model 32 and the control from the display form controller 362.

The combining unit 368 superimposes the heat map image on the input medical image to generate the composite image for display. The combining unit 368 may further generate the composite image in which the superimposition information is superimposed on the input medical image.

The display controller 38 generates a display signal required for display output to the display device 324 and executes display control of the display device 324. The composite image generated by the combining unit 368 is displayed on the display device 324 via the display controller 38.

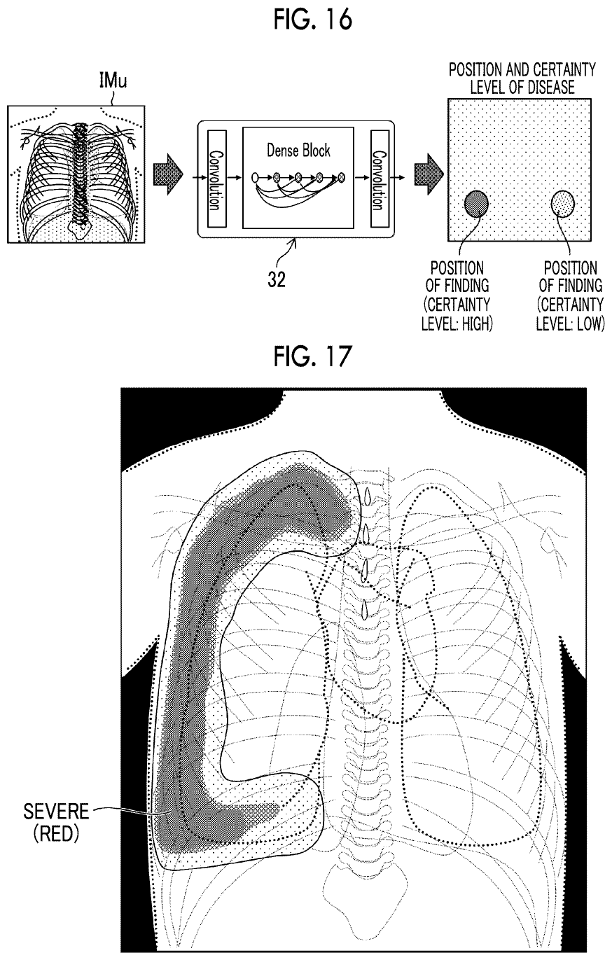

FIG. 16 is an explanatory diagram showing an example of an image processing method executed by using the third machine learning model 32 implemented in the image processing device 30. It should be noted that the third machine learning model 32 shown in FIG. 16 is a trained model that has been trained using the ground truth data generated by Example 1 of the label generation method shown in FIG. 3.

The processor 302 of the image processing device 30 executes processing of inputting the unknown medical image IMu as the processing target to the third machine learning model 32 and calculating the position and the certainty level of the disease for the unknown medical image IMu by using the third machine learning model 32.

In addition, the processor 302 executes processing of changing the display form of the disease in accordance with the value of the certainty level of the disease for the medical image IMu acquired by using the third machine learning model 32, and displaying the processing result on the display device 324.

### Type of Disease as Detection Target

The disease as the target detected from the plain chest X-ray image is not limited to pleural effusion, and may be, for example, pneumothorax, pulmonary tuberculosis, or an appropriate combination thereof.

### Control Example 1 of Display Form according to Certainty Level

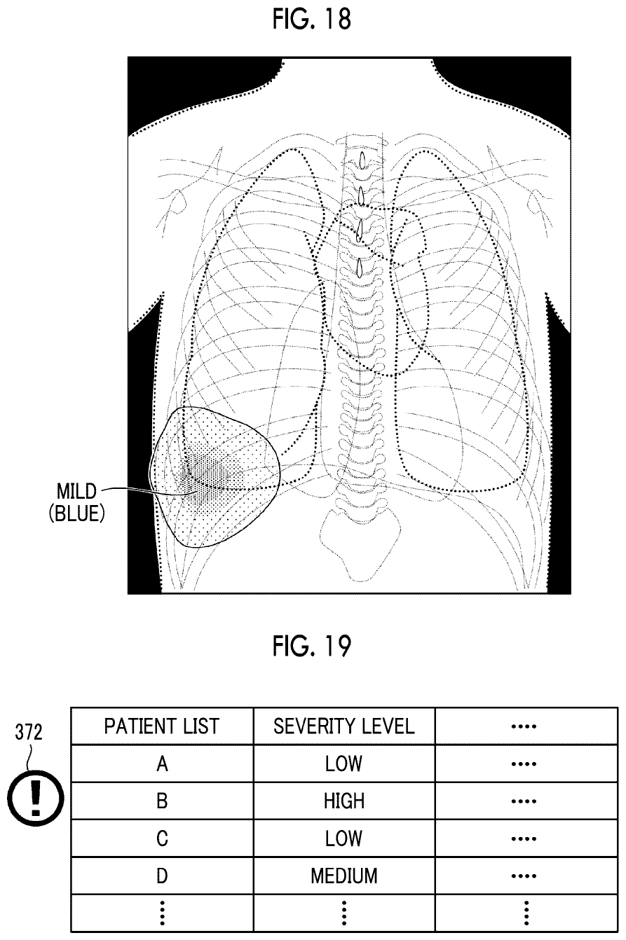

FIGS. 17 and 18 show examples of the composite images displayed on the display device 324 as the processing results of the third machine learning model 32. FIGS. 17 and 18 show examples of a form in which information is displayed in a different color in accordance with the certainty level calculated by the third machine learning model 32. FIGS. 17 and 18 show an example of the composite image in which the heat map image that visualizes the position and the certainty level of the disease estimated by the third machine learning model 32 is superimposed on the medical image.

FIG. 17 shows an image example displayed in a case in which a severe disease is detected from the medical image, and FIG. 18 shows an image example displayed in a case in which a mild disease is detected from the medical image. In the heat map image superimposed on the medical image, for example, a pixel having a relatively high certainty level is colored relatively red, and a pixel having a relatively low certainty level is colored relatively purple. It is preferable that the color is displayed in a more visually appealing manner as the value of the certainty level is larger.

For example, “red” may be displayed for a region of the severe disease, and “blue” may be displayed for a region of the mild disease. As shown in FIG. 17, in a case in which the severe disease is detected, that is, in a case in which the disease with a high certainty level is detected, the heat map image indicating the region of the detected disease is displayed in red.

On the other hand, as shown in FIG. 18, in a case in which the mild disease is detected, that is, in a case in which the disease with a low certainty level is detected, the heat map image showing the region of the detected disease is displayed in blue (or purple).

It should be noted that a correspondence relationship between the value of the certainty level and the color in the heat map image is not limited to this example, and various definitions can be made.

### Control Example 2 of Display Form according to Certainty Level

FIG. 19 is an explanatory diagram showing another example in which the display form is changed in accordance with the certainty level of the disease. FIG. 19 shows a display example of the examination list that provides information on the severity level of the disease indicated by the certainty level calculated by the third machine learning model 32. As shown in FIG. 19, an alert 372 is displayed for the data of the patient in which the disease with a high severity level is recognized in the examination list in which the examination results of the plurality of patients are displayed in a list. In the example of FIG. 19, the alert 372 is assigned to the row (record) of the data of the patient B. By displaying the alert 372, the doctor can easily identify the patient who needs an emergency treatment.

### Type of Medical Image

In the embodiment described above, a case has been described in which the plain chest X-ray image is used as an example of the medical image IM, but the medical image as the target is not limited to the plain chest X-ray image, and various medical images captured by various medical apparatuses (modalities) such as a CT image, an MR image captured using an MRI apparatus, an ultrasound image, a PET image, or an endoscopic image can be targeted. The image targeted by the technology of the present disclosure is not limited to the two-dimensional image, and may be a three-dimensional image.

### Hardware Configuration of Each Processing Unit

The hardware structures of the processing units that execute various types of processing, such as the data acquisition unit 130, the disease detection unit 140, the anatomical structure extraction unit 141, the disease position conversion unit 142, the 3D image analysis unit 143, the certainty level label conversion unit 14, the label data conversion unit 145, and the association unit 16 in the label generation device 10, the learning processing unit 24, the loss calculation unit 26, and the parameter update unit 28 in the machine learning device 20, and the medical image acquisition unit 31, the disease detection unit 34, the display information generation unit 36, the display form controller 362, the heat map image generation unit 364, the superimposition information generation unit 366, and the combining unit 368, and the display controller 38 in the image processing device 30 according to the embodiment described above, are various processors, for example, as shown below.

The various processors include a CPU that is a general-purpose processor that executes the program and that functions as the various processing units, a GPU, a programmable logic device (PLD) that is a processor of which a circuit configuration can be changed after manufacture, such as a field programmable gate array (FPGA), and a dedicated electric circuit that is a processor of which a circuit configuration is designed for exclusive use in order to execute specific processing, such as an application specific integrated circuit (ASIC).

One processing unit may be configured by one of these various processors or two or more processors of the same type or different types. One processing unit may be configured by, for example, a plurality of FPGAs, a combination of a CPU and an FPGA, or a combination of a CPU and a GPU. A plurality of the processing units may also be configured by one processor. As an example in which the plurality of processing units are configured by one processor, first, there is a form in which one processor is configured by a combination of one or more CPUs and software, and this processor functions as the plurality of processing units, as typified by a computer, such as a client or a server. Second, there is a form in which a processor is used, which achieves the functions of the entire system including the plurality of processing units with one integrated circuit (IC) chip, as typified by a system on a chip (SoC) or the like. In this way, various processing units are configured by one or more of the various processors described above, as the hardware structure.

Further, the hardware structure of these various processors is, more specifically, an electric circuit (circuitry) in which circuit elements, such as semiconductor elements, are combined.

### Program for Causing Computer to Operate

A program for causing the computer to achieve a part or all of the processing functions in each of the label generation device 10, the machine learning device 20, and the image processing device 30 described in the embodiment described above can be recorded on a computer-readable medium that is a non-transitory information storage medium such as an optical disk, a magnetic disk, or a semiconductor memory, and the program can be provided through the information storage medium.

Instead of the aspect in which the program is stored in such a tangible non-transitory computer-readable medium and provided, a program signal can be provided as a download service by using an electric communication line, such as the Internet.

Further, a part or all of the processing functions in each of the devices described above may be achieved by cloud computing, or can be provided as software as a service (Saas).

### Advantages of Embodiment of Present Disclosure

According to the embodiment of the present disclosure described above, the following effects can be obtained.
(1) The label generation device 10 can efficiently generate the ground truth label that can contribute to the generation of the second machine learning model 22 by using the definitive diagnosis examination information in which the disease position is indefinite or the disease position is recorded in granularity different from the desired region division granularity.
(2) The machine learning device 20 can generate the trained second machine learning model 22 that estimates the position of the disease and the certainty level corresponding to the severity level from the medical image through the machine learning using the ground truth data GT generated by the label generation device 10.
(3) The image processing device 30 can provide the information indicating the position and the certainty level of the disease in the unknown medical image IMu in a form that is easy for the doctor to intuitively understand, by using the third machine learning model 32, which is the trained model generated by the machine learning device 20.

### Others

The present disclosure is not limited to the embodiment described above, and various modifications can be made without departing from the gist of the technical idea of the present disclosure.

### EXPLANATION OF REFERENCES

1: system
4: examination information management device
6: storage device
10: label generation device
12: first machine learning model
13: analysis model
14: certainty level label conversion unit
16: association unit
20: machine learning device
22: second machine learning model
24: learning processing unit
26: loss calculation unit
28: parameter update unit
30: image processing device
31: medical image acquisition unit
32: third machine learning model
34: disease detection unit
36: display information generation unit
38: display controller
40: electric communication line
102: processor
104: computer-readable medium
106: communication interface
108: input/output interface
110: bus
112: memory
114: storage
122: input device
124: display device
130: data acquisition unit
132: medical image acquisition unit
134: definitive diagnosis examination information acquisition unit
140: disease detection unit
141: anatomical structure extraction unit
142: disease position conversion unit
143: 3D image analysis unit
145: label data conversion unit
147: anatomical structure extraction unit
148: disease detection unit
149: pleural effusion amount calculation unit
150: data storage unit
162: medical image acquisition program
164: definitive diagnosis examination information acquisition program
170: disease detection program
171: anatomical structure extraction program
172: disease position constraint program
173: disease position conversion program
183: 3D image analysis program
184: certainty level label conversion program
185: label data conversion program
186: association program
187: ground truth data storage processing program
188: display control program
190: anatomical structure extraction program
191: disease detection program
192: pleural effusion amount calculation program
194: look-up table
202: processor
204: computer-readable medium
206: communication interface
208: input/output interface
210: bus
212: memory
214: storage
222: input device
224: display device
230: machine learning program
232: data acquisition program
236: loss calculation program
238: optimizer
240: display control program
250: training data storage unit
302: processor
304: computer-readable medium
306: communication interface
308: input/output interface
310: bus
312: memory
314: storage
322: input device
324: display device
332: medical image acquisition program
334: disease detection program
336: display form control program
338: heat map image generation program
340: superimposition information generation program
342: combination program
344: display control program
362: display form controller
364: heat map image generation unit
366: superimposition information generation unit
368: combining unit
372: alert
AS1, AS2: anatomical structure information
DD, DD1, DD2, DD2_1, DD2_2: definitive diagnosis examination information
DP1, DP2: disease position data
DS: data set
FP1a, FP1b: region
GT, GT1, GT1_2, GT2, GTj: ground truth data
IM, IM1, IM2, IMj, IMu: medical image
PD: paired data
PRj: output data
SM, SM1, SM2: saliency map
TDS: training data set
S10 to S22: steps of Example 1 of label generation method
S30 to S50: steps of Example 3 of label generation method
S60 to S68: steps of machine learning method

What is claimed is:

1. A label generation method comprising:
causing one or more first processors to execute:
a step of acquiring one or more candidate positions of a disease in a first division unit from a first medical image;
a step of acquiring diagnostic information, for the first medical image, in which a position of the disease is indefinite or the position of the disease is specified in a second division unit;
a step of converting the diagnostic information into a certainty level label corresponding to a severity level of the disease;
a step of associating a certainty level of the disease corresponding to the certainty level label with the candidate positions of the disease acquired from the first medical image; and
a step of acquiring a ground truth label, which is generated by the association, of the position and the certainty level of the disease with respect to the first medical image.

2. The label generation method according to claim 1,
wherein in the step of acquiring the ground truth label,
the one or more first processors acquire the ground truth label of the position and the certainty level of the disease in the first division unit or the second division unit.

3. The label generation method according to claim 1, further comprising:
causing the one or more first processors to execute:
a step of acquiring anatomical structure information from the first medical image,
wherein in the step of associating the certainty level of the disease with the candidate positions of the disease,
the position of the disease is constrained to be located within a desired anatomical structure specified from the anatomical structure information.

4. The label generation method according to claim 1,
wherein the diagnostic information is a three-dimensional examination image, and
the step of converting the diagnostic information into the certainty level label includes
a step of recognizing an anatomical structure from the three-dimensional examination image,
a step of recognizing the position of the disease from the three-dimensional examination image, and
a step of calculating the certainty level label of the disease for each anatomical structure from the recognized anatomical structure and the recognized position of the disease.

5. The label generation method according to claim 1,
wherein the diagnostic information is sputum examination information including an examination result of a sputum examination, and
the step of converting the diagnostic information into the certainty level label includes
a step of calculating the certainty level label of the disease based on an amount of bacteria collected in the sputum examination.

6. The label generation method according to claim 1,
wherein in the step of acquiring the one or more candidate positions of the disease,
a saliency map of the disease is calculated by using a first machine learning model that has been trained in advance.

7. The label generation method according to claim 6,
wherein in the step of associating the certainty level of the disease with the candidate positions of the disease,
the certainty level label is weighted by a value of the saliency map.

8. The label generation method according to claim 1,
wherein the first medical image is a chest X-ray image, a computed tomography image, or a magnetic resonance image.

9. The label generation method according to claim 1,
wherein at least one of pleural effusion, pneumothorax, or pulmonary tuberculosis is targeted as the disease.

10. A trained model generation method comprising:
causing one or more second processors to execute:
a step of training a second machine learning model through machine learning using training data including the ground truth label generated by the label generation method according to claim 1,
wherein the trained second machine learning model is generated, which has been trained to receive an input of a second medical image and output the position and the certainty level of the disease with respect to the second medical image.

11. The trained model generation method according to claim 10,
wherein the certainty level label of the disease is represented by a continuous value, and
in the step of training the second machine learning model, the certainty level of the disease is regression-predicted from the first medical image by the second machine learning model.

12. The trained model generation method according to claim 10,
wherein the certainty level label of the disease is represented by a discrete value, and
in the step of training the second machine learning model, the certainty level of the disease is classification-predicted from the first medical image by the second machine learning model.

13. An image processing method comprising:
causing one or more third processors to execute:
a step of calculating, by using the trained second machine learning model generated by the trained model generation method according to claim 10, the position and the certainty level of the disease with respect to the second medical image.

14. The image processing method according to claim 13, further comprising:
causing the one or more third processors to execute:
a step of changing a display form of the disease in accordance with a value of the certainty level of the disease with respect to the second medical image.

15. A label generation device comprising:
one or more first processors,
wherein the one or more first processors execute:
processing of acquiring one or more candidate positions of a disease in a first division unit from a first medical image;
processing of acquiring diagnostic information, for the first medical image, in which a position of the disease is indefinite or the position of the disease is specified in a second division unit;
processing of converting the diagnostic information into a certainty level label corresponding to a severity level of the disease;
processing of associating a certainty level of the disease corresponding to the certainty level label with the candidate positions of the disease acquired from the first medical image; and
processing of acquiring a ground truth label, which is generated by the processing of associating, of the position and the certainty level of the disease with respect to the first medical image.

16. A machine learning device comprising:
one or more second processors,
wherein the one or more second processors
execute processing of training a second machine learning model through machine learning using training data including the ground truth label generated by the label generation method according to claim 1, and
the second machine learning model is trained such that the second machine learning model receives an input of a second medical image and outputs the position and the certainty level of the disease in the second medical image.

17. An image processing device comprising:
one or more third processors,
wherein the one or more third processors
execute processing of calculating, by using the trained second machine learning model generated by the trained model generation method according to claim 11, the position and the certainty level of the disease with respect to the second medical image.

18. A non-transitory, computer-readable tangible recording medium which records thereon a program for causing, when read by a computer, the computer to execute the label generation method according to claim 1.

19. A non-transitory, computer-readable tangible recording medium which records thereon a program for causing, when read by a computer, the computer to execute the trained model generation method according to claim 10.

20. A non-transitory, computer-readable tangible recording medium which records thereon a program for causing, when read by a computer, the computer to execute the image processing method according to claim 13.
